(19) United States
(12) Patent Application Publication
(10) Pub. No.: US 2025/0134824 A1
(43) Pub. Date: May 1, 2025

(54) LOW-DOSE LYOPHILIZED RNA VACCINES AND METHODS FOR PREPARING AND USING THE SAME

(71) Applicant: GLAXOSMITHKLINE BIOLOGICALS SA, Rixensart (BE)

(72) Inventors: Rushit LODAYA, Rockville, MD (US); Patrick POHLHAUS, Rockville, MD (US)

(73) Assignee: GLAXOSMITHKLINE BIOLOGICALS SA, Rixensart (BE)

(21) Appl. No.: 18/683,531

(22) PCT Filed: Aug. 16, 2022
(86) PCT No.: PCT/IB2022/057663

§ 371 Date: Feb. 14, 2024

Related U.S. Application Data
(60) Provisional application No. 63/233,401, filed on Aug. 16, 2021.

(51) Int. Cl. A61K 9/51; A61K 9/19; A61K 39/00

(52) U.S. Cl. CPC A61K 9/5192; A61K 9/19; A61K 2039/53; A61K 2039/54; A61K 2039/545

(57) ABSTRACT

Compositions and methods are provided for stabilization of RNA encapsulated by lipid nanoparticles during lyophilization. The compositions and methods involve the use of empty lipid nanoparticles to stabilize the lyophilized composition. These techniques may be used to prevent the need for cold chain storage and may also simplify the procedure at the clinic for reconstituting the vaccine to prepare an injectable composition.

### CROSS REFERENCE TO RELATED APPLICATION

This application claims the benefit of U.S. Application No. 63/233,401, filed Aug. 16, 2021, the entirety of which is incorporated by reference herein.

### TECHNICAL FIELD

The present invention relates to the field of lyophilized RNA pharmaceutical formulations and methods of making and using said lyophilized RNA pharmaceutical formulations.

### BACKGROUND

A major challenge for mRNA vaccines is efficient delivery to the target site while avoiding degradation and clearance from the body. To overcome this barrier, lipid nanoparticles (LNPs), in which RNA is encapsulated in a liquid core of a LNP, have emerged as one of the most efficient delivery vehicles for RNA (see EP 2 591 103). The LNP protects the RNA from degradation and enables its cellular uptake and intracellular release.

However, a disadvantage of lipid nanoparticle encapsulated RNA (RNA-LNP) is its instability, (e.g., due to susceptibility to hydrolysis). To avoid such degradation, RNA-LNP is typically stored at minus 20° C. to minus 80° C. under RNase free conditions.

For distribution of RNA-LNP containing therapeutics, the pharmaceutical composition containing the RNA-LNP is subject to cold chain storage, in which it is typically maintained at a temperature ranging from −20° C. to −80° C. during its lifecycle. However, maintaining cold chain storage has a number of disadvantages, including cost, complexity of distribution, and potential loss of product. In some cases, failure to maintain cold chain storage, e.g., due to refrigeration failure, electrical outages, etc., may necessitate discarding of the pharmaceutical composition. Additionally, once reaching respective endpoints, pharmaceutical compositions that cannot be stored at the designated temperature must be administered within a prescribed timeframe or discarded.

Accordingly, it is desirable to improve long term stability of pharmaceutical compositions comprising RNA as well as prevent and/or reduce the need for cold chain storage, with the goal to preserve RNA entrapment in lipid nanoparticles while maintaining transfection potency after lyophilization.

### BRIEF SUMMARY

This invention is directed to a lyophilized low-dose RNA immunogenic composition or vaccine that can be easily reconstituted just prior to use to prepare an injectable low-dose vaccine composition, kits for making said immunogenic composition or vaccine and methods of using the immunogenic compositions and vaccines and kits. The invention also relates to a method of administering a low-dose vaccine to a subject. The invention also relates to a vaccine composition for use in eliciting an immune response in a patient to which the vaccine composition is administered, an injectable vaccine composition for use in eliciting an immune response in a patient to which the vaccine composition is administered, and use of a vaccine composition for the manufacture of a medicament for use in inducing an acceptable immune response in a subject, wherein the medicament is prepared to be administered by injection.

In one embodiment, the invention is directed to a method of preparing a low-dose lyophilized RNA immunogenic composition, comprising: forming a mixed aqueous composition comprising RNA containing lipid nanoparticles (RNA-LNPs), wherein the RNA encodes at least one immunogen, and empty lipid nanoparticles (empty LNPs); and lyophilizing the mixed aqueous composition. The low-dose immunogenic composition can be used to make a vaccine composition comprising the immunogenic composition.

In another embodiment, the invention relates to a low-dose lyophilized immunogenic composition, comprising: RNA containing lipid nanoparticles (RNA-LNPs), wherein the RNA encodes at least one immunogen; and empty lipid nanoparticles (empty LNPs)

### DETAILED DESCRIPTION

Applicant herein provides embodiments for methods of preparing a low-dose lyophilized RNA immunogenic composition and embodiments for a vaccine composition, a low-dose immunogenic composition, and a low-dose vaccine kit.

In one embodiment, the present invention is directed to a lyophilized low-dose SAM-LNP drug product that can be reconstituted at the dosing site with only a single dilution or no further dilution, after reconstitution, to produce an injectable vaccine composition where the concentration of SAM in the composition is as low as 1 and 2 μg/mL of RNA such as SAM. This enables a strategy to dose 0.5 μg and 1 μg (for a dose of 0.5 ml), respectively, without any further dilution at the dosing site, thus simplifying the dosing process. For example, if the concentration in the dried vaccine (upon reconstitution with a pre-determined volume of liquid) will be ½ μg/mL, a 0.5 mL human dose would have 0.5/1 μg dose of SAM in the vaccine. This would enable low-dose vaccine without the need for further dilution after reconstitution. Some processes and formulations limit the development of stable lyophilized SAM-LNPs below the concentration of 60 μg/mL. Although the reasons why it is not possible to make low-dose vaccines that do not need further dilution after reconstitution are not fully understood, it is possible that the freezing and/or thawing processes during lyophilization damage the LNPs, especially at low concentrations. It seems that the more the concentration of the SAM-LNP is reduced, the more damage is done to the LNPs during lyophilization. It is possible that by including or adding empty particles as ‘decoy’ particles, these decoy particles prevent the SAM-LNP particles to be negatively impacted during the lyophilization process or at least reduces the negative impact on the particles that would otherwise be observed in the absence of the decoy particles. Thus, in some respects, the empty LNPs can be considered as excipients that have a stabilizing effect on the lyophilized vaccine.

In another embodiment, the present invention is directed to a low-dose vaccine that can be reconstituted with limited loss in % encapsulation. The limited loss in encapsulation upon reconstitution is made possible by, prior to lyophilization, making sure that there are a limited number of empty LNPs in the RNA-LNP formulation. This idea of intentionally making sure that empty LNPs are present is counter to the general understanding in the art that the presence of empty LNPs is not desired, and is to be avoided. It has been surprisingly found that the inclusion of a limited number of empty LNPs in the RNA-LNP formulation, prior to lyophilization, allows the lyophilized LNP to be reconstituted with limited loss in CQAs, especially limited loss in % encapsulation. The inventors have discovered that a high loss in % encapsulation, distinctly observed at low concentrations of SAM-LNP after lyophilization, can be recovered, and in some cases, improved compared to lyophilized SAM-LNP control formulation at a higher concentration such as 60 μg/mL.

In one embodiment, empty LNPs are mixed with an excess of RNA-LNPs to produce a low-dose vaccine composition comprising a predetermined amount of empty LNPs.

The present invention relates to lyophilized RNA pharmaceutical formulations and methods of making and using said lyophilized low-dose RNA pharmaceutical formulations. The pharmaceutical compositions of the invention preferably comprise lipid nanoparticles (LNPs) wherein at least some of the LNPs contain mRNA therein. The pharmaceutical composition, prior to administration to a patient, is in a lyophilized form and it is reconstituted with a liquid for reconstitution (such as sterile water for injection) and optionally further diluted with a liquid prior to administration to a patient by injection. The present invention is also directed to methods for making all the above formulations. The present invention also provides a low-dose pharmaceutical composition comprising lipid nanoparticles formulated for lyophilization, a vaccine, a kit, and a method of lyophilization of the low-dose pharmaceutical composition. The present invention provides novel low-dose pharmaceutical compositions formulated for lyophilization, including a lipid nanoparticle encapsulating RNA, a primary sugar, and a buffer with one or more optional additional components. The present invention provides a novel method of lyophilization of the low-dose pharmaceutical composition, wherein the method comprises a freezing step, a primary drying step, and a secondary drying step. The present invention provides a novel use of a pharmaceutical low-dose composition for stabilizing lipid nanoparticle encapsulated RNA during lyophilization. The present invention provides a novel use of the inventive low-dose pharmaceutical composition and/or method in the manufacture of a medicament, an immunogenic composition, a vaccine, and/or kit.

The present invention is particularly suited for low-dose mRNA vaccine compositions such as self-amplifying mRNA (SAM) LNP vaccine compositions containing a SAM/LNP drug product at a concentration of less than 60 μg/mL of SAM. In some applications, SAM-LNP vaccine is 1 μg or less based on ongoing clinical trials with SARS-CoV2 SAM-LNP vaccine. This invention describes development of lyophilized SAM-LNP drug product at low concentrations i.e. 1 and 2 μg/mL, that enable a strategy to dose 0.5 μg and 1 μg, respectively, without any further dilution after reconstitution at the dosing site. Thus, in accordance with the present invention it is possible to simply reconstitute the low-dose vaccine with an appropriate liquid and further dilutions after reconstitution, such as 1 or 2 further dilutions, are not necessary. Thus, with the present invention, additional dilution steps can be eliminated thus making the dosing at the clinical site simpler. Prior art processes and products limit the development of stable lyophilized SAM-LNPs below the concentration of 60 μg/mL.

Lyophilized low-dose vaccines prepared in accordance with the present invention ideally have little or no loss in CQAs (Critical Quality Attributes) or at least have acceptable levels of loss in CQAs. Some of the CQAs that should be maintained at the same level or an acceptable level when an RNA is reconstituted include, for example, one or more of % encapsulation, size, polydispersity index (PDI), in vitro relative potency and purity. Avoiding loss in CQAs during constitution is particularly difficult with RNA vaccines where the RNA is present at low doses. For example, it is expected that the dose of a SARS-CoV2 SAM-LNP vaccine for clinical trials may be on the order of a few (3) micrograms or less, such as 3, 2, 1 or 0.5 μgs of SAM.

This invention describes proof of concept that upon addition of empty LNPs to low concentration SAM-LNPs, the high loss in % encapsulation, distinctly observed at low concentrations of SAM, after lyophilization, can be recovered, and in some cases, improved compared to lyophilized SAM-LNP control formulation at 60 μg/mL.

### The RNA

Any type of RNA that encodes an immunogen is contemplated for use in accordance with the present invention. The RNA can be a self-replication mRNA such as SAM or can be non-replicating mRNA.

mRNA

Messenger RNA (mRNA) can direct the cellular machinery of a subject to produce proteins. mRNA may be circular or branched, but will generally be linear. mRNA as used herein are preferably provided in purified or substantially purified form, i.e. substantially free from proteins (e.g., enzymes), other nucleic acids (e.g. DNA and nucleoside phosphate monomers), and the like, generally being at least about 50% pure (by weight), and usually at least 90% pure, such as at least 95%, 96%, 97% or at least 98% pure.

mRNA may be prepared in many ways, e.g., by chemical synthesis in whole or in part, by digesting longer nucleic acids using nucleases (e.g., restriction enzymes), by joining shorter nucleic acids or nucleotides (e.g., using ligases or polymerases), from genomic or cDNA libraries, etc. In particular, mRNA may be prepared enzymatically using a DNA template.

The term mRNA as used herein includes conventional mRNA or mRNA analogs, such as those containing modified backbones or modified bases (e.g., pseudouridine, or the like). mRNA, may or may not have a 5′ cap.

The mRNA comprises a sequence which encodes at least one antigen or immunogen. Typically, the nucleic acids of the invention will be in recombinant form, i.e. a form which does not occur in nature. For example, the mRNA may comprise a sequence encoding an antigen and/or a control sequence such as a promoter or an internal ribosome entry site (IRES). In another example, the mRNA may comprise one or more heterologous nucleic acid sequences (e.g., another sequence encoding another antigen and/or another control sequence such as a promoter or an IRES in addition to the sequence encoding the antigen).

Alternatively, or in addition, the sequence or chemical structure of the nucleic acid may be modified compared to a naturally-occurring sequence which encodes the antigen. The sequence of the nucleic acid molecule may be modified, e.g., to increase the efficacy of expression or replication of the nucleic acid, or to provide additional stability or resistance to degradation.

mRNA may also be codon optimised. In some embodiments, mRNA may be codon optimised for expression in human cells. “Codon optimised” is intended to refer to modification(s) with respect to codon usage, which may increase translation efficacy and/or half-life of the nucleic acid.

A poly A tail (e.g., of about 30 adenosine residues or more) may be attached to the 3′ end of the mRNA to increase its half-life.

The mRNA may also include a poly-A polymerase recognition sequence (e.g. AAUAAA) near its 3′ end.

The 5′ end of the mRNA may be capped, for example, with a modified ribonucleotide having the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) comprising or consisting of mRNA triphosphatase, guanylyl-transferase and guanine-7-methyltransferase, which catalyzes the construction of N7-monomethylated cap 0 structures). Cap 0 structure plays an important role in maintaining the stability and translational efficacy of the mRNA molecule. The 5′ cap of the mRNA molecule may be further modified by a 2′-O-Methyltransferase which results in the generation of a cap 1 structure (m7Gppp [m2′-O]N), which may further increase translation efficacy.

mRNA may comprise one or more nucleotide analogs or modified nucleotides. As used herein, “nucleotide analog” or “modified nucleotide” refers to a nucleotide that contains one or more chemical modifications (e.g., substitutions) in or on the nitrogenous base of the nucleoside (e.g., cytosine (C), thymine (T) or uracil (U)), adenine (A) or guanine (G)). A nucleotide analog can contain further chemical modifications in or on the sugar moiety of the nucleoside (e.g., ribose, modified ribose, six-membered sugar analog, or open-chain sugar analog), or the phosphate. The preparation of nucleotides and modified nucleotides and nucleosides are well-known in the art (see, U.S. Pat. Nos. 4,373,071, 4,458,066, 4,500,707, 4,668,777, 4,973,679, 5,047,524, 5,132,418, 5,153,319, 5,262,530, 5,700,642). Many modified nucleosides and modified nucleotides are commercially available.

Modified nucleobases (chemical modifications) which can be incorporated into modified nucleosides and nucleotides and be present in the mRNA molecules include: m5C (5-methylcytidine); m5U (5-methyluridine); m6A (N6-methyladenosine); s2U (2-thiouridine); Um (2′-O-methyluridine); m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); m′1 (1-methylinosine); m′1m (1,2′-O-dimethylinosine); m3C (3-methylcytidine); Cm (2′-O-methylcytidine); s2C (2-thiocytidine); ac4C (N4-acetylcytidine); f5C (5-fonnylcytidine); m5Cm (5,2-O-dimethylcytidine); ac4Cm (N4-acetyl-2-O-methylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethy 1 aminomethyl-2-L-O-methyl uridine); cmnm5s2U (5-carboxymethylaminomethyl-2-thiouridine); m62A (N6,N6-dimethyladenosine); Im (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,2′-O-dimethyladenosine); m62Am (N6,N6,0-2-trimethyladenosine); m2′7G (N2,7-dimethylguanosine); m2′2′7G (N2,N2,7-trimethylguanosine); m3Um (3,2′-O-dimethyluridine); m5D (5-methyldihydrouridine); f5Cm (5-formyl-2′-O-methylcytidine); m1Gm (1,2′-O-dimethylguanosine); m′Am (1,2-O-dimethyl adenosine) irinomethyluridine); tm5s2U (S-taurinomethyl-2-thiouridine)); iniG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C1-C6)-alkyluracil, 5-methyluracil, 5-(C2-C6)-alkenyluracil, 5-(C2-C6)-alkynyluracil, 5-(hydroxymethyl)uracil, 5-chlorouracil, 5-fluorouracil, 5-bromouracil, 5-hydroxycytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Many of these modified nucleobases and their corresponding ribonucleosides are available from commercial suppliers.

### Modified Nucleotides

In some embodiments, the modified nucleotides comprise: pseudouridine; N1-methylpseudouridine; N1-ethylpseudouridine; 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6,N6 (dimethyl)adenine; N6-cis-hydroxy-isopentenyl-adenosine; .alpha.-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; .alpha.-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine: 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine: 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; .alpha.-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester, 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester, 5-carboxymethylaminomethyl-2′-O-methyluridine; 5-carboxymethylaminomethyl-2-thiouridine; 5-carboxymethylaminomethyl-2-thiouridine; 5-caboxymethylaminomethyluridine; 5-carboxymethylaminomethyluridine; 5-Carbamoylmethyluridine TP; 5-methoxycaeoonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; N1-ethyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; .alpha.-thio-uridine; 1 (aminoalkylamino-carbonylethylenyl)-2(thio)-pseudouridine; 1 (aminoalkylaminocarbonylethylenyl)-2,4-(dithio)pseudouridine; 1 (aminoalkylaminocarbonylethylenyl)-4 (thio)pseudouridine; 1 (aminoalkylaminocarbonylethylenyl)-pseudouridine; 1 (aminocazbonylethylenyl)-2(thio)-pseudouridine; 1 (aminocarbonylethylenyl)-2,4-(dithio)pseudouridine; 1 (aminocarbonylethylenyl)-4 (thio)pseudouridine; 1 (aminocarbonylethylenyl)-pseudouridine; 1 substituted 2(thio)-pseudouridine; 1 substituted 2,4-(dithio)pseudouridine; 1 substituted 4 (thio)pseudouridine; 1 substituted pseudouridine; 1-(aminoalkylamino-carbonylethylenyl)-2-(thio)-pseudouridine; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouridine; 2′ deoxy uridine; 2′ fluorouridine; 2-(thio)uracil; 2,4-(dithio)psuedouracil; 2′ methyl, 2′amino, 2′azido, 2′fluoro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouridine; 4-(thio)pseudouridine; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouridine; 5-(alkyl)-2,4 (dithio)pseudouridine; 5-(alkyl)-4 (thio)pseudouridine; 5-(alkyl)pseudouridine; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouridine; 5-(methyl)-2,4 (dithio)pseudouridine; 5-(methyl)-4 (thio)pseudouridine; 5-(methyl)pseudouridine; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouridine; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (.+-.)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1-(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouri-dine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2-(2-ethoxy)-ethoxy)-ethoxy}-ethoxy]-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluoro-cytidine; 2′ methyl, 2′amino, 2′azido, 2′fluoro-adenine; 2′methyl, 2′amino, 2′azido, 2′fluoro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; or N6-(19-Amino-pentaoxanonadecyl)adenosine TP.

In some embodiments, the adenosine-substitutable modified nucleotides comprise: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6,N6 (dimethyl)adenine; N6-cis-hydroxy-isopentenyl-adenosine; .alpha.-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluoro-cytidine; 2′ methyl, 2′amino, 2′azido, 2′fluoro-adenine; 2′methyl, 2′amino, 2′azido, 2′fluoro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; or N6-(19-Amino-pentaoxanonadecyl)adenosine TP.

In some embodiments, the uridine-substitutable modified nucleotides or the thymidine-substitutable modifified nucleotides comprise: pseudouridine; N1-methylpseudouridine; N1-ethylpseudouridine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester, 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester, 5-carboxymethylaminomethyl-2′-O-methyluridine; 5-carboxymethylaminomethyl-2-thiouridine; 5-carboxymethylaminomethyl-2-thiouridine; 5-caboxymethylaminomethyluridine; 5-carboxymethylaminomethyluridine; 5-Carbamoylmethyluridine TP; 5-methoxycaeoonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; N1-ethyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; .alpha.-thio-uridine; 1 (aminoalkylamino-carbonylethylenyl)-2(thio)-pseudouridine; 1 (aminoalkylaminocarbonylethylenyl)-2,4-(dithio)pseudouridine; 1 (aminoalkylaminocarbonylethylenyl)-4 (thio)pseudouridine; 1 (aminoalkylaminocarbonylethylenyl)-pseudouridine; 1 (aminocazbonylethylenyl)-2(thio)-pseudouridine; 1 (aminocarbonylethylenyl)-2,4-(dithio)pseudouridine; 1 (aminocarbonylethylenyl)-4 (thio)pseudouridine; 1 (aminocarbonylethylenyl)-pseudouridine; 1 substituted 2(thio)-pseudouridine; 1 substituted 2,4-(dithio)pseudouridine; 1 substituted 4 (thio)pseudouridine; 1 substituted pseudouridine; 1-(aminoalkylamino-carbonylethylenyl)-2-(thio)-pseudouridine; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouridine; 2′ deoxy uridine; 2′ fluorouridine; 2-(thio)uracil; 2,4-(dithio)psuedouracil; 2′ methyl, 2′amino, 2′azido, 2′fluoro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouridine; 4-(thio)pseudouridine; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouridine; 5-(alkyl)-2,4 (dithio)pseudouridine; 5-(alkyl)-4 (thio)pseudouridine; 5-(alkyl)pseudouridine; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouridine; 5-(methyl)-2,4 (dithio)pseudouridine; 5-(methyl)-4 (thio)pseudouridine; 5-(methyl)pseudouridine; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouridine; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (.+-.)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1-(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouri-dine TP; 1-{3-[2-(2-Aminoethoxy)-ethoxy]-propionyl}pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2-(2-ethoxy)-ethoxy)-ethoxy}-ethoxy]-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluoro-cytidine; 2′ methyl, 2′amino, 2′azido, 2′fluoro-adenine; 2′methyl, 2′amino, 2′azido, 2′fluoro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-cytidine TP; 2′-OH-ara-uridine TP TP; or 5-(2-carbomethoxyvinyl)uridine TP.

In some embodiments, the cytosine-substitutable modified nucleotides comprise 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; .alpha.-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine: 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine: 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 2′fluoro-cytidine; or 2′-OH-ara-cytidine TP.

In some embodiments, the modified nucleotides comprise: 7-methylguanosine; N2,2′-0-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-0-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; .alpha.-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; or 2′-OH-ara-guanosine TP.

The mRNA may encode more than one antigen. For example, the mRNA encoding an antigen protein may encode only the antigen or may encode additional proteins. Each antigen and additional protein(s) may be under the control of different regulatory elements. Alternatively, the antigen and additional proteins may be under the control of the same regulatory element. Where at least two additional proteins are encoded, some of the antigen and additional proteins may be under the control of the same regulatory element and some may be under the control of different regulatory elements.

mRNA may be non-replicating or may be replicating, also known as self-replicating. A self-replicating mRNA molecule may be an alphavirus-derived mRNA replicon. mRNA amplification can also be achieved by the provision of a non-replicating mRNA encoding an antigen in conjunction with a separate mRNA encoding replication machinery.

Self-replicating RNA molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen, or may be transcribed to provide further transcripts with the same sense as the delivered RNA which are translated to provide in situ expression of the antigen. The overall result of this sequence of transcriptions is a huge amplification in the number of the introduced replicon RNAs and so the encoded antigen becomes a major polypeptide product of the cells.

Suitable alphavirus replicons can use a replicase from a Sindbis virus, a Semliki forest virus, an eastern equine encephalitis virus, a Venezuelan equine encephalitis virus, etc. Mutant or wild-type virus sequences can be used, e.g., the attenuated TC83 mutant of VEEV has been used in replicons, (see WO2005/113782).

In certain embodiments, the self-replicating RNA molecule described herein encodes (i) an RNA-dependent RNA polymerase which can transcribe RNA from the self-replicating RNA molecule and (ii) an antigen. The polymerase can be an alphavirus replicase, e.g., comprising one or more of alphavirus proteins nsP1, nsP2, nsP3 and nsP4.

Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the antigen/immunogen of interest, such that the subgenomic transcript encodes the immunogen rather than the structural alphavirus virion proteins.

Thus, a self-replicating RNA molecule useful with the invention may have two open reading frames. The first (5′) open reading frame encodes a replicase; the second (3′) open reading frame encodes an antigen. In some embodiments the RNA may have additional (e.g., downstream) open reading frames, e.g., to encode further antigens or to encode accessory polypeptides.

In certain embodiments, the self-replicating RNA molecule disclosed herein has a 5′ cap (e.g., a 7-methylguanosine). This cap can enhance in vivo translation of the RNA. In some embodiments, the 5′ sequence of the self-replicating RNA molecule must be selected to ensure compatibility with the encoded replicase.

A self-replicating RNA molecule may have a 3′ poly-A tail. It may also include a poly-A polymerase recognition sequence (e.g., AAUAAA) near its 3′ end.

Self-replicating RNA molecules can have various lengths, but are typically 5000 to 25000 nucleotides long, such as 8000 to 15000 nucleotides long, for example 9000 to 12000 nucleotides long. Self-replicating RNA molecules will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA.

In another embodiment, a self-replicating RNA may comprise two separate RNA molecules, each comprising a nucleotide sequence derived from an alphavirus: one RNA molecule comprises an RNA construct for expressing alphavirus replicase, and one RNA molecule comprises an RNA replicon that can be replicated by the replicase in trans. In some embodiments, the RNA construct for expressing alphavirus replicase comprises a 5′-cap. See WO 2017/162265.

The self-replicating RNA can conveniently be prepared by in vitro transcription (IVT). IVT can use a (cDNA) template created and propagated in plasmid form in bacteria, or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the self-replicating RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

A self-replicating RNA can include (in addition to any 5′ cap structure) one or more nucleotides having a modified nucleobase. An RNA used with the invention ideally includes only phosphodiester linkages between nucleosides, but in some embodiments it can contain phosphoramidate, and/or methylphosphonate linkages.

The self-replicating RNA molecule may encode a single heterologous polypeptide antigen (i.e. the antigen) or, optionally, two or more heterologous polypeptide antigens linked together in a way that each of the sequences retains its identity (e.g., linked in series) when expressed as an amino acid sequence. The heterologous polypeptides generated from the self-replicating RNA may then be produced as a fusion polypeptide or engineered in such a manner to result in separate polypeptide or peptide sequences.

The self-replicating RNA molecules described herein may be engineered to express multiple nucleotide sequences, from two or more open reading frames, thereby allowing co-expression of proteins, such as one, two or more antigens (e.g. one, two or more protein(s) together with cytokines or other immunomodulators, which can enhance the generation of an immune response). Such a self-replicating RNA molecule might be particularly useful, for example, in the production of various gene products (e.g., proteins) at the same time, for example, as a bivalent or multivalent vaccine.

If desired, the self-replicating RNA molecules can be screened or analyzed to confirm their therapeutic and prophylactic properties using various in vitro or in vivo testing methods that are known to those of skill in the art. For example, vaccines comprising self-replicating RNA molecule can be tested for their effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a self-replicating RNA molecule that encodes an antigen can be tested. In addition, T helper cell differentiation can be analyzed by measuring proliferation or production of TH1 (IL-2 and IFN-7) and/or TH2 (IL-4 and IL-5) cytokines by ELISA or directly in CD4+ T cells by cytoplasmic cytokine staining and flow cytometry.

Self-replicating RNA molecules that encode an antigen can also be tested for ability to induce humoral immune responses, as evidenced, for example, by induction of B cell production of antibodies specific for the antigen of interest. These assays can be conducted using, for example, peripheral B lymphocytes from immunized individuals. Such assay methods are known to those of skill in the art. Other assays that can be used to characterize the self-replicating RNA molecules can involve detecting expression of the encoded antigen by the target cells. For example, FACS can be used to detect antigen expression on the cell surface or intracellularly. Another advantage of FACS selection is that one can sort for different levels of expression; sometimes-lower expression may be desired. Other suitable methods for identifying cells which express a particular antigen involve panning using monoclonal antibodies on a plate or capture using magnetic beads coated with monoclonal antibodies.

A non-replicating mRNA will typically contain 10000 bases or fewer, especially 8000 bases or fewer, in particular 5000 bases or fewer. A replicating mRNA will typically contain 25000 bases or fewer, especially 20000 bases or fewer, in particular 15000 bases or fewer. A replicating mRNA may contain 5000 to 25000 nucleotides, such as 8000 to 15000 nucleotides, for example 9000 to 12000 nucleotides.

In one embodiment the mRNA is non-replicating mRNA. In a second embodiment the mRNA is replicating mRNA.

A preferred self-replicating RNA molecule thus encodes (i) a RNA-dependent RNA polymerase which can transcribe RNA from the self-replicating RNA molecule and (ii) an immunogen. The polymerase can be an alphavirus replicase e.g. comprising one or more of alphavirus proteins nsP1, nsP2, nsP3 and nsP4.

A self-replicating RNA can include one or more modified pyrimidine nucleobases, such as pseudouridine and/or 5-methylcytosine residues. In some embodiments, however, the RNA includes no modified nucleobases, and may include no modified nucleotides i.e. all of the nucleotides in the RNA are standard A, C, G and U ribonucleotides (except for any 5′ cap structure, which may include a 7′-methylguanosine). In other embodiments, the RNA may include a 5′ cap comprising a 7′-methylguanosine, and the first 1, 2 or 3 5′ ribonucleotides may be methylated at the 2′ position of the ribose.

### Amount of RNA in a Low-Dose Vaccine Composition

The RNA is present in the low-dose vaccine composition is present in a low-dose as defined in this application. A single low dose of mRNA may be as low as 0.001 but less than 60 μg, for example 0.1 to less than 60 μg. A replicating mRNA dose (such as a SAM dose) will usually be lower than a non-replicating mRNA dose encoding the same immunogen. A non-replicating mRNA dose may, for example, be 1 to less than 60 μg, such as 10 to less than 60 μg. Where the composition (or container such as a vial holding the composition) contains multiple doses, the composition can contain the above amounts of RNA times the number of doses. In other words, in accordance with the present invention, a container holding the composition can contain a single low-dose or multiple low-doses. The number of low-doses that can be in the container is discussed in other parts of this application.

In one embodiment, the present invention relates to including empty LNPs in a low-dose vaccine composition over a wide range of possible doses, including the above doses.

Generally speaking, RNA-LNPs, such as mRNA-LNPs or SAM-LNPs, are formulated based on RNA concentration and the total amount (mass) of RNA needed for each dose. The concentration of RNA (such as mRNA or SAM) pre-lyophilization and post-lyophilization may be about the same. Thus, for example, with a SAM-LNP concentration of 1 μg/mL, 700 μL of SAM-LNP can be filled into a lyophilization vial and the lyophilization vial should theoretically contain 0.7 μg of RNA. During lyophilization, the volume of the RNA-LNP product, such as SAM-LNP lyophilized product, may expand (relative to the volume of the pre-lyophilized aqueous composition) because of the lyophilization process. Post lyophilization, the lypophlized product such as lyophilized powder and/or cake described just above may be reconstituted in 646 μL of water for injection (WFI). Upon reconstitution, the concentration of SAM-LNP is expected to be about the same the volume pre-lyophilization (and usually is around that upon testing using Ribogreen). When 0.5 mL is dosed (500 μL—human dose), it is expected that the dose will be 0.5 μg of SAM in the SAM-LNP.

In another embodiment, the present invention is directed to improving one or more characteristics of a low-dose RNA-LNP, mRNA-LNP or SAM-LNP composition by including empty LNPs in the composition. Unless otherwise indicated, the amount or concentration of RNA, mRNA or SAM in the composition is the amount of RNA, mRNA or SAM that is presumed to be encapsulated in the a RNA-LNP composition and does not include any amount that is outside of the LNP, which, in any event, is expected to be only a very small amount, such as 5% or less, less than 5%, less than 4%, less than 3%, less than 2%, less than 1% or less than 0.5%, if any at all. The RNA-LNP, mRNA-LNP or SAM-LNP composition may be substantially or completely free of RNA, mRNA or SAM-LNP that is outside of LNPs. In accordance with this embodiment of the invention, a “low-dose” vaccine composition is an effective amount of RNA, mRNA or SAM of less than 60 μg of RNA, mRNA or SAM, preferably less than 30 μg of RNA, mRNA or SAM, more preferably less than 10 μg of RNA, mRNA or SAM, even more preferably less than 5 μg of RNA, mRNA or SAM and most preferably less than 3 μg of RNA, mRNA or SAM. More specifically, the low-dose can be an effective amount up to less than 2.5 μg or less than 3 μg of RNA, mRNA or SAM, such as 0.5 to 2 μg of SAM, 0.75 to 2 μg of SAM or 1 to 2 μg of SAM.

As one example, it is expected that the dose of a SARS-CoV2 SAM-LNP vaccine for clinical trials may be on the order of a few (3) micrograms or less, such as 3, 2, 1 or 0.5 μgs of SAM (RNA).

### The Immunogen/Antigen

RNA molecules encapsulated within a LNP encode a polypeptide immunogen. After administration of the RNA, the immunogen is translated in vivo and can elicit an immune response in the recipient. The immunogen may elicit an immune response against a bacterium, a virus, a fungus or a parasite (or, in some embodiments, against an allergen; and in other embodiments, against a tumor antigen). The immune response may comprise an antibody response (usually including IgG) and/or a cell-mediated immune response. The polypeptide immunogen will typically elicit an immune response which recognises the corresponding bacterial, viral, fungal or parasite (or allergen or tumour) polypeptide, but in some embodiments the polypeptide may act as a mimotope to elicit an immune response which recognizes a bacterial, viral, fungal or parasite saccharide. The immunogen will typically be a surface polypeptide e.g. an adhesin, a hemagglutinin, an envelope glycoprotein, a spike glycoprotein, etc.

RNA molecules can encode a single polypeptide immunogen or multiple polypeptides. Multiple immunogens can be presented as a single polypeptide immunogen (fusion polypeptide) or as separate polypeptides. If immunogens are expressed as separate polypeptides then one or more of these may be provided with an upstream IRES or an additional viral promoter element. Alternatively, multiple immunogens may be expressed from a polyprotein that encodes individual immunogens fused to a short autocatalytic protease (e.g., foot-and-mouth disease virus 2A protein).

Unlike Johanning et al. (1995) Nucleic Acids Res 23:1495-1501 and El Ouahabi et al. (1996) FEBS Letts 380:108-12, the RNA encodes an immunogen. For the avoidance of doubt, the invention does not encompass RNA which encodes a firefly luciferase or which encodes a fusion protein of E. coli β-galactosidase or which encodes a green fluorescent protein (GFP). Also, the RNA is not total mouse thymus RNA.

In some embodiments the immunogen elicits an immune response against one of these bacteria:
Neisseria meningitidis: useful immunogens include, but are not limited to, membrane proteins such as adhesins, autotransporters, toxins, iron acquisition proteins, and factor H binding protein. A combination of three useful polypeptides is disclosed in Giuliani et al. (2006) Proc Natl Acad Sci USA 103(29):10834-9.
Streptococcus pneumoniae: useful polypeptide immunogens are disclosed in WO 2009/016515. These include, but are not limited to, the RrgB pilus subunit, the beta-N-acetyl-hexosaminidase precursor (spr0057), spr0096, General stress protein GSP-781 (spr2021, SP2216), serine/threonine kinase StkP (SP1732), and pneumococcal surface adhesin PsaA.
Streptococcus pyogenes: useful immunogens include, but are not limited to, the polypeptides disclosed in WO 02/34771 and WO 2005/032582.
Moraxella catarrhalis.
Bordetella pertussis: Useful pertussis immunogens include, but are not limited to, pertussis toxin or toxoid (PT), filamentous haemagglutinin (FHA), pertactin, and agglutinogens 2 and 3.
Staphylococcus aureus: Useful immunogens include, but are not limited to, the polypeptides disclosed in WO 2010/119343, such as a hemolysin, esxA, esxB, ferrichrome-binding protein (sta006) and/or the sta011 lipoprotein.
Clostridium tetani: the typical immunogen is tetanus toxoid.
Cornynebacterium diphtheriae: the typical immunogen is diphtheria toxoid.
Haemophilus influenzae: Useful immunogens include, but are not limited to, the polypeptides disclosed in WO 2006/110413 and WO 2005/111066.
Pseudomonas aeruginosa
Streptococcus agalactiae: useful immunogens include, but are not limited to, the polypeptides disclosed in WO 2006/089264.
Chlamydia trachomatis: Useful immunogens include, but are not limited to, PepA, LcrE, ArtJ, DnaK, CT398, OmpH-like, L7/L12, OmcA, AtoS, CT547, Eno, HtrA and MurG (e.g. as disclosed in WO 2005/002619. LcrE (see, WO 2006/138004) and HtrA (see WO 2009/109860) are two preferred immunogens.
Chlamydia pneumoniae: Useful immunogens include, but are not limited to, the polypeptides disclosed in WO 02/02606.
Helicobacter pylori: Useful immunogens include, but are not limited to, CagA, VacA, NAP, and/or urease WO 03/018054.
Escherichia coli: Useful immunogens include, but are not limited to, immunogens derived from enterotoxigenic E. coli (ETEC), enteroaggregative E. coli (EAggEC), diffusely adhering E. coli (DAEC), enteropathogenic E. coli (EPEC), extraintestinal pathogenic E. coli (ExPEC) and/or enterohemorrhagic E. coli (EHEC). ExPEC strains include uropathogenic E. coli (UPEC) and meningitis/sepsis-associated E. coli (MNEC). Useful UPEC polypeptide immunogens are disclosed in WO 2006/091517 and WO 2008/020330. Useful MNEC immunogens are disclosed in WO 2006/089264. A useful immunogen for several E. coli types is AcfD (see WO 2009/104092).
Bacillus anthracis
Yersinia pestis: Useful immunogens include, but are not limited to, those disclosed in WO 2009/031043 and WO 2007/049155.
Staphylococcus epidermis
Clostridium perfringens or Clostridium botulinums
Legionella pneumophila
Coxiella burnetii
Brucella, such as B. abortus, B. canis, B. melitensis, B. neotomae, B. ovis, B. suis, B. pinnipediae.
Francisella, such as F. novicida, F. philomiragia, F. tularensis.
Neisseria gonorrhoeae
Treponema pallidum
Haemophilus ducreyi
Enterococcus faecalis or Enterococcus faecium
Staphylococcus saprophyticus
Yersinia enterocolitica
Mycobacterium tuberculosis
Rickettsia
Listeria monocytogenes
Vibrio cholerae
Salmonella typhi
Borrelia burgdorferi
Porphyromonas gingivalis
Klebsiella

In some embodiments the immunogen elicits an immune response against one of these viruses:
Orthomyxovirus: Useful immunogens can be from an influenza A, B or C virus, such as the hemagglutinin, neuraminidase or matrix M2 proteins. Where the immunogen is an influenza A virus hemagglutinin it may be from any subtype e.g. H1, H2, H3, H4, H5, H6, H7, H8, H9, H10, H11, H12, H13, H14, H15 or H16.
Paramyxoviridae viruses: Viral immunogens include, but are not limited to, those derived from Pneumoviruses (e.g. respiratory syncytial virus, RSV), Rubulaviruses (e.g. mumps virus), Paramyxoviruses (e.g. parainfluenza virus), Metapneumoviruses and Morbilliviruses (e.g. measles).
Poxviridae: Viral immunogens include, but are not limited to, those derived from Orthopoxvirus such as Variola vera, including but not limited to, Variola major and Variola minor.
Picornavirus: Viral immunogens include, but are not limited to, those derived from Picornaviruses, such as Enteroviruses, Rhinoviruses, Heparnavirus, Cardioviruses and Aphthoviruses. In one embodiment, the enterovirus is a poliovirus e.g. a type 1, type 2 and/or type 3 poliovirus. In another embodiment, the enterovirus is an EV71 enterovirus. In another embodiment, the enterovirus is a coxsackie A or B virus.
Bunyavirus: Viral immunogens include, but are not limited to, those derived from an Orthobunyavirus, such as California encephalitis virus, a Phlebovirus, such as Rift Valley Fever virus, or a Nairovirus, such as Crimean-Congo hemorrhagicfever virus.
Heparnavirus: Viral immunogens include, but are not limited to, those derived from a Heparnavirus, such as hepatitis A virus (HAV).
Filovirus: Viral immunogens include, but are not limited to, those derived from a filovirus, such as an Ebola virus (including a Zaire, Ivory Coast, Reston or Sudan ebolavirus) or a Marburg virus.
Togavirus: Viral immunogens include, but are not limited to, those derived from a Togavirus, such as a Rubivirus, an Alphavirus, or an Arterivirus. This includes rubella virus.
Flavivirus: Viral immunogens include, but are not limited to, those derived from a Flavivirus, such as Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus.
Pestivirus: Viral immunogens include, but are not limited to, those derived from a Pestivirus, such as Bovine viral diarrhea (BVDV), Classical swine fever (CSFV) or Border disease (BDV).
Hepadnavirus: Viral immunogens include, but are not limited to, those derived from a Hepadnavirus, such as Hepatitis B virus. A composition can include hepatitis B virus surface antigen (HBsAg).
Other hepatitis viruses: A composition can include an immunogen from a hepatitis C virus, delta hepatitis virus, hepatitis E virus, or hepatitis G virus.
Rhabdovirus: Viral immunogens include, but are not limited to, those derived from a Rhabdovirus, such as a Lyssavirus (e.g. a Rabies virus) and Vesiculovirus (VSV).
Caliciviridae: Viral immunogens include, but are not limited to, those derived from Calciviridae, such as Norwalk virus (Norovirus), and Norwalk-like Viruses, such as Hawaii Virus and Snow Mountain Virus.
Coronavirus: Viral immunogens include, but are not limited to, those derived from a SARS coronavirus, avian infectious bronchitis (IBV), Mouse hepatitis virus (MHV), and Porcine transmissible gastroenteritis virus (TGEV). The coronavirus immunogen may be a spike polypeptide.

Viral immunogens include, but are not limited to, those derived from a SARS coronavirus, COVID-19 (including various strains thereof), avian infectious bronchitis (IBV), Mouse hepatitis virus (MHV), and Porcine transmissible gastroenteritis virus (TGEV). The coronavirus immunogen may be a spike polypeptide. See, e.g., Wrapp et al. (2020) “Cryo-EM structure of the 2019-nCoV spike in the prefusion conformation.” Science, 367:1260-1263.
Coronavirus spike polypeptides or proteins, including fragments and modifications thereof, are known in the art and are included as immunogens.
Retrovirus: Viral immunogens include, but are not limited to, those derived from an Oncovirus, a Lentivirus (e.g. HIV-1 or HIV-2) or a Spumavirus.
Reovirus: Viral immunogens include, but are not limited to, those derived from an Orthoreovirus, a Rotavirus, an Orbivirus, or a Coltivirus.
Parvovirus: Viral immunogens include, but are not limited to, those derived from Parvovirus B19.
Herpesvirus: Viral immunogens include, but are not limited to, those derived from a human herpesvirus, such as, by way of example only, Herpes Simplex Viruses (HSV) (e.g. HSV types 1 and 2), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8).
Papovaviruses: Viral immunogens include, but are not limited to, those derived from Papillomaviruses and Polyomaviruses. The (human) papillomavirus may be of serotype 1, 2, 4, 5, 6, 8, 11, 13, 16, 18, 31, 33, 35, 39, 41, 42, 47, 51, 57, 58, 63 or 65 e.g. from one or more of serotypes 6, 11, 16 and/or 18.
Adenovirus: Viral immunogens include those derived from adenovirus serotype 36 (Ad-36).

In some embodiments, the immunogen elicits an immune response against a virus which infects fish, such as: infectious salmon anemia virus (ISAV), salmon pancreatic disease virus (SPDV), infectious pancreatic necrosis virus (IPNV), channel catfish virus (CCV), fish lymphocystis disease virus (FLDV), infectious hematopoietic necrosis virus (IHNV), koi herpesvirus, salmon picorna-like virus (also known as picorna-like virus of atlantic salmon), landlocked salmon virus (LSV), atlantic salmon rotavirus (ASR), trout strawberry disease virus (TSD), coho salmon tumor virus (CSTV), or viral hemorrhagic septicemia virus (VHSV).

Fungal immunogens may be derived from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme; or from Aspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; the less common are Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

In some embodiments the immunogen elicits an immune response against a parasite from the Plasmodium genus, such as P. falciparum, P. vivax, P. malariae or P. ovale. Thus the invention may be used for immunising against malaria. In some embodiments the immunogen elicits an immune response against a parasite from the Caligidae family, particularly those from the Lepeophtheirus and Caligus genera e.g. sea lice such as Lepeophtheirus salmonis or Caligus rogercresseyi.

In some embodiments the immunogen elicits an immune response against: pollen allergens (tree-, herb, weed-, and grass pollen allergens); insect or arachnid allergens (inhalant, saliva and venom allergens, e.g. mite allergens, cockroach and midges allergens, hymenopthera venom allergens); animal hair and dandruff allergens (from e.g. dog, cat, horse, rat, mouse, etc.); and food allergens (e.g. a gliadin). Important pollen allergens from trees, grasses and herbs are such originating from the taxonomic orders of Fagales, Oleales, Pinales and platanaceae including, but not limited to, birch (Betula), alder (Alnus), hazel (Corylus), hornbeam (Carpinus) and olive (Olea), cedar (Cryptomeria and Juniperus), plane tree (Platanus), the order of Poales including grasses of the genera Lolium, Phleum, Poa, Cynodon, Dactylis, Holcus, Phalaris, Secale, and Sorghum, the orders of Asterales and Urticales including herbs of the genera Ambrosia, Artemisia, and Parietaria. Other important inhalation allergens are those from house dust mites of the genus Dermatophagoides and Euroglyphus, storage mite e.g. Lepidoglyphys, Glycyphagus and Tyrophagus, those from cockroaches, midges and fleas e.g. Blatella, Periplaneta, Chironomus and Ctenocepphalides, and those from mammals such as cat, dog and horse, venom allergens including such originating from stinging or biting insects such as those from the taxonomic order of Hymenoptera including bees (Apidae), wasps (Vespidea), and ants (Formicoidae).

In some embodiments the immunogen is a tumor antigen selected from: (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors; (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT; (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), mammaglobin, alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer); (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma); (e) prostate associated antigens such as PAP, PSA, PSMA, PSH-P1, PSM-P1, PSM-P2, associated with e.g., prostate cancer; (f) immunoglobulin idiotypes (associated with myeloma and B cell lymphomas, for example). In certain embodiments, tumor immunogens include, but are not limited to, p15, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, TPS, and the like.

Lipid Nanoparticles (RNA, mRNA or SAM Carriers)

A range of carrier systems have been described which encapsulate RNA, mRNA or SAM in order to facilitate mRNA delivery and consequent expression of encoded antigens as compared to mRNA which is not encapsulated. The present invention may utilise any suitable carrier system. Particular mRNA carrier systems of note are further described below.

Lipid nanoparticles (LNPs) are non-virion liposome particles in which mRNA can be encapsulated. LNP delivery systems and methods for their preparation are known in the art (see Liposomes: Methods and Protocols, Volume 1: Pharmaceutical Nanocarriers: Methods and Protocols. (ed. Weissig). Humana Press, 2009. ISBN 160327359X and Liposome Technology, volumes I, II & III. (ed. Gregoriadis). Informa Healthcare, 2006) and involves mixing (i) an ethanolic solution of the lipids (ii) an aqueous solution of the nucleic acid and (iii) buffer, followed by mixing, equilibration, dilution and purification. Preferred liposomes of the invention are obtainable by this mixing process. RNA is preferably encapsulated within the liposomes, and the liposome forms an outer layer around an aqueous RNA-containing core. This encapsulation has been found to protect RNA from RNase digestion. The particles can include some external mRNA (e.g. on the surface of the particles), but desirably at least half of the RNA (and suitably at least 85%, especially at least 95%, 96%, 97%, 98% or 99%, such as all of it) is encapsulated.

Various amphiphilic lipids can form bilayers in an aqueous environment to encapsulate a RNA-containing aqueous core as a liposome. These lipids can have an anionic, cationic or zwitterionic hydrophilic head group. Some phospholipids are anionic whereas other are zwitterionic and others are cationic. Suitable classes of phospholipid include, but are not limited to, phosphatidylethanolamines, phosphatidylcholines, phosphatidylserines, and phosphatidylglycerols, and some useful phospholipids are listed in Table 1 below.

### Table 1: Useful Phospholipids

DDPC 1,2-Didecanoyl-sn-Glycero-3-phosphatidylcholine
DEPA 1,2-Dierucoyl-sn-Glycero-3-Phosphate
DEPC 1,2-Erucoyl-sn-Glycero-3-phosphatidylcholine
DEPE 1,2-Dierucoyl-sn-Glycero-3-phosphatidylethanolamine
DEPG 1,2-Dierucoyl-sn-Glycero-3[Phosphatidyl-rac-(1-glycerol . . . )
DLOPC 1,2-Linoleoyl-sn-Glycero-3-phosphatidylcholine
DLPA 1,2-Dilauroyl-sn-Glycero-3-Phosphate
DLPC 1,2-Dilauroyl-sn-Glycero-3-phosphatidylcholine
DLPE 1,2-Dilauroyl-sn-Glycero-3-phosphatidylethanolamine
DLPG 1,2-Dilauroyl-sn-Glycero-3[Phosphatidyl-rac-(1-glycerol . . . )
DLPS 1,2-Dilauroyl-sn-Glycero-3-phosphatidylserine
DMG 1,2-Dimyristoyl-sn-glycero-3-phosphoethanolamine
DMPA 1,2-Dimyristoyl-sn-Glycero-3-Phosphate
DMPC 1,2-Dimyristoyl-sn-Glycero-3-phosphatidylcholine
DMPE 1,2-Dimyristoyl-sn-Glycero-3-phosphatidylethanolamine
DMPG 1,2-Myristoyl-sn-Glycero-3[Phosphatidyl-rac-(1-glycerol . . . )
DMPS 1,2-Dimyristoyl-sn-Glycero-3-phosphatidylserine
DOPA 1,2-Dioleoyl-sn-Glycero-3-Phosphate
DOPC 1,2-Dioleoyl-sn-Glycero-3-phosphatidylcholine
DOPE 1,2-Dioleoyl-sn-Glycero-3-phosphatidylethanolamine
DOPG 1,2-Dioleoyl-sn-Glycero-3[Phosphatidyl-rac-(1-glycerol . . . )
DOPS 1,2-Dioleoyl-sn-Glycero-3-phosphatidylserine
DPPA 1,2-Dipalmitoyl-sn-Glycero-3-Phosphate
DPPC 1,2-Dipalmitoyl-sn-Glycero-3-phosphatidylcholine
DPPE 1,2-Dipalmitoyl-sn-Glycero-3-phosphatidylethanolamine
DPPG 1,2-Dipalmitoyl-sn-Glycero-3[Phosphatidyl-rac-(1-glycerol . . . )
DPPS 1,2-Dipalmitoyl-sn-Glycero-3-phosphatidylserine
DPyPE1,2-diphytanoyl-sn-glycero-3-phosphoethanolamine
DSPA 1,2-Distearoyl-sn-Glycero-3-Phosphate
DSPC 1,2-Distearoyl-sn-Glycero-3-phosphatidylcholine
DSPE 1,2-Diostearpyl-sn-Glycero-3-phosphatidylethanolamine
DSPG 1,2-Distearoyl-sn-Glycero-3[Phosphatidyl-rac-(1-glycerol . . . )
DSPS 1,2-Distearoyl-sn-Glycero-3-phosphatidylserine
EPC Egg-PC
HEPC Hydrogenated Egg PC
HSPC High purity Hydrogenated Soy PC
HSPC Hydrogenated Soy PC
LYSOPC MYRISTIC 1-Myristoyl-sn-Glycero-3-phosphatidylcholine
LYSOPC PALMITIC 1-Palmitoyl-sn-Glycero-3-phosphatidylcholine
LYSOPC STEARIC 1-Stearoyl-sn-Glycero-3-phosphatidylcholine
Milk Sphingomyelin MPPC 1-Myristoyl,2-palmitoyl-sn-Glycero 3-phosphatidylcholine
MSPC 1-Myristoyl,2-stearoyl-sn-Glycero-3-phosphatidylcholine
PMPC 1-Palmitoyl,2-myristoyl-sn-Glycero-3-phosphatidylcholine
POPC 1-Palmitoyl,2-oleoyl-sn-Glycero-3-phosphatidylcholine
POPE 1-Palmitoyl-2-oleoyl-sn-Glycero-3-phosphatidylethanolamine
POPG 1,2-Dioleoyl-sn-Glycero-3[Phosphatidyl-rac-(1-glycerol) . . . ]
PSPC 1-Palmitoyl,2-stearoyl-sn-Glycero-3-phosphatidylcholine
SMPC 1-Stearoyl,2-myristoyl-sn-Glycero-3-phosphatidylcholine
SOPC 1-Stearoyl,2-oleoyl-sn-Glycero-3-phosphatidylcholine
SPPC 1-Stearoyl,2-palmitoyl-sn-Glycero-3-phosphatidylcholine

Useful cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids are DPPC, DOPC and dodecylphosphocholine. The lipids can be saturated or unsaturated. The use of at least one unsaturated lipid for preparing liposomes is preferred. If an unsaturated lipid has two tails, both tails can be unsaturated, or it can have one saturated tail and one unsaturated tail.

LNP formulated mRNA may be prepared comprising mRNA, cationic lipid, and other helper lipids. Liposomal particles can, for example, be formed of a mixture of zwitterionic, cationic and anionic lipids which can be saturated or unsaturated, for example; DSPC (zwitterionic, saturated), DlinDMA (cationic, unsaturated), and/or DMG (anionic, saturated). Preferred LNPs for use with the invention include an amphiphilic lipid which can form liposomes, optionally in combination with at least one cationic lipid (such as DOTAP, DSDMA, DODMA, DLinDMA, DLenDMA, etc.). A mixture of DSPC, DlinDMA, PEG-DMG and cholesterol is particularly effective. Other useful LNPs are described in WO 2011/076807; WO 2012/006372, WO 2012/006376; WO2012/030901; WO 2012/031046; WO 2012/031043; WO 2012/006378; WO 2013/033563; WO 2013/006825; WO 2014/136086; WO 2015/095340; WO 2015/095346; and WO 2016/037053. In some embodiments, the LNPs are RVO1 liposomes, see Liposome Technology, volumes I, II & III. (ed. Gregoriadis). Informa Healthcare, 2006; and Geall et al. (2012) PNAS USA. September 4; 109(36): 14604-9.

Liposomes can be formed from a single lipid or from a mixture of lipids. A mixture may comprise (i) a mixture of anionic lipids (ii) a mixture of cationic lipids (iii) a mixture of zwitterionic lipids (iv) a mixture of anionic lipids and cationic lipids (v) a mixture of anionic lipids and zwitterionic lipids (vi) a mixture of zwitterionic lipids and cationic lipids or (vii) a mixture of anionic lipids, cationic lipids and zwitterionic lipids. Similarly, a mixture may comprise both saturated and unsaturated lipids. For example, a mixture may comprise DSPC (zwitterionic, saturated), DlinDMA (cationic, unsaturated), and/or DMG (anionic, saturated). Where a mixture of lipids is used, not all of the component lipids in the mixture need to be amphiphilic e.g. one or more amphiphilic lipids can be mixed with cholesterol.

LNP can, for example, be formed of a mixture of (i) a PEG-modified lipid (ii) a non-cationic lipid (iii) a sterol (iv) an ionisable cationic lipid. Alternatively, LNP can for example be formed of a mixture of (i) a PEG-modified lipid (ii) a non-cationic lipid (iii) a sterol (iv) a non-ionisable cationic lipid.

In certain embodiments, the LNP may comprise an RNA or mRNA, such as but not limited to SAM, a cationic lipid, a zwitterionic lipid such as phosphatidylcholine, a sterol such as cholesterol, and optionally a PEGylated lipid. In certain embodiments, the LNP may comprise RNA or an mRNA, such as but not limited to SAM, an ionisable cationic lipid, a zwitterionic lipid such as phosphatidylcholine, a sterol such as cholesterol, and optionally a PEGylated lipid. In other embodiments, the LNP may comprise DSPC, DlinDMA, PEG-DMG and cholesterol. In still other embodiments, the LNP may comprise DSPC, 2,5-bis((9Z,12Z)-octadeca-9,12-dien-1-yloxy)benzyl 4-(dimethylamino)butanoate)(“RV39”), PEG and cholesterol. See WO2016/037053. Example 1, describing RV39. In some embodiments, the cation-ionizable lipid is:

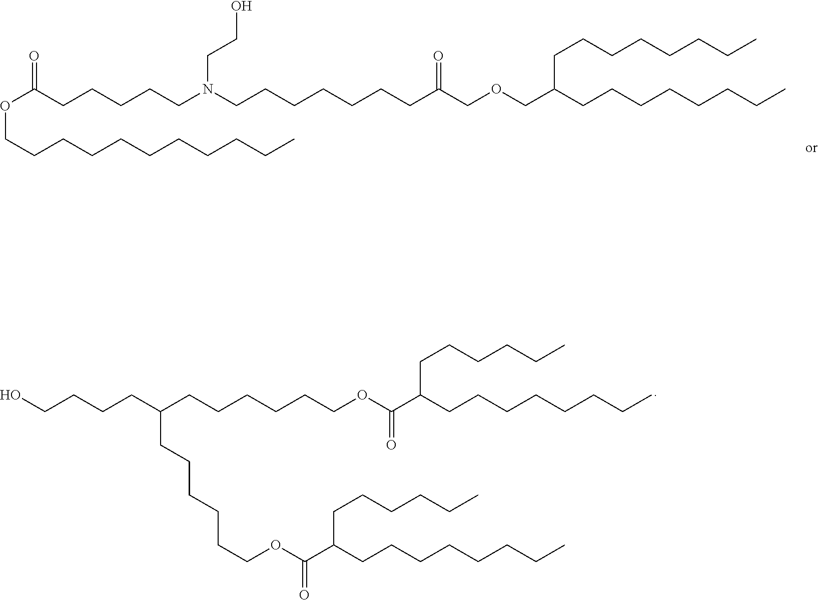 [SMILES: CCCCCCCCCCCOC(=O)CCCCCN(CCO)CCCCCCCC(=O)COCC(CCCCCCCC)CCCCCCCC ; CCCCCCCCC(CCCCCC)C(=O)OCCCCCCC(CCCCO)CCCCCCOC(=O)C(CCCCCC)CCCCCCCC]

In some embodiments, the PEG-conjugated lipid comprises 2-[(polyethylene glycol)-2000]-N,N-ditetradecylacetamide or 1,2-dimyristoyl-rac-glycero-3-methoxypolyethylene glycol-2000. In 2-[(polyethylene glycol)-2000]-N,N-ditetradecylacetamide or 1,2-dimyristoyl-rac-glycero-3-methoxypolyethylene glycol-2000, the “2000” represents the median molecular weight in Daltons of the PEG. In some embodiments, the PEG-conjugated lipid comprises 1,2-dimyristoyl-sn-glycero-2-phosphoethanolamine-N-[methoxy(polyethylene glycol)]. In some embodiments, the PEG-conjugated lipid comprises 1,2-dimyristoyl-rac-glycerol-3-methoxypolyethylene glycol. In some embodiments, the PEG-conjugated lipid comprises:

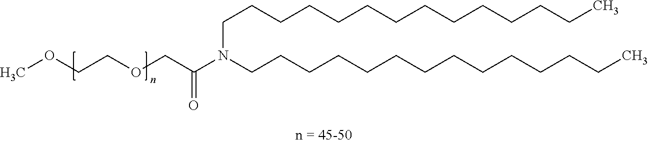

The PEG-modified lipid may comprise a PEG molecule with a molecular weight of 10000 Da or less, especially 5000 Da or less, in particular 3000 Da, such as 2000 Da or less. Examples of PEG-modified lipids include PEG-distearoyl glycerol, PEG-dipalmitoyl glycerol and PEG-dimyristoyl glycerol. The PEG-modified lipid is typically present at around 0.5 to 15 molar %.

The non-cationic lipid may be a neutral lipid, such as 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE) and sphingomyelin (SM). The non-cationic lipid is typically present at around 5 to 25 molar %.

The sterol may be cholesterol. The sterol is typically present at around 25 to 55 molar % b.

A range of suitable ionizable cationic lipids are known in the art, which are typically present at around 20 to 60 molar %.

The ratio of RNA to lipid can be varied (see, for example, WO 2013/006825). “N:P ratio” refers to the molar ratio of protonatable nitrogen atoms in the cationic lipids (typically solely in the lipid's headgroup) to phosphates in the RNA. The ratio of nucleotide (N) to phospholipid (P) can be in the range of, e.g., 1N:1P to 20N:1P, 1N:1P to 10N:1P, 2N:1P to 8N:1P, 2N:1P to 6N:1P or 3N:1P to 5N:1P. The ratio of nucleotide (N) to phospholipid (P) can be in the range of, e.g., 1N:1P, 2N:1P, 3N:1P, 4N:1P, 5N:1P, 6N:1P, 7N:1P, 8N:1P, 9N:1P, or 10N:1P. Alternatively or additionally, the ratio of nucleotide (N) to phospholipid (P) is 4N:1P.

General information on LNP compositions is provided in the following references: Liposome Technology, volumes I, II & III. (ed. Gregoriadis). Informa Healthcare, 2006; WO 2012/006359 and WO 2017/070620.

LNP delivery systems, and methods for their preparation are described in Geall et al. (2012) PNAS USA. September 4; 109(36): 14604-9 (LNP delivery system).

LNPs are typically 50 to 200 um in diameter (Z-average). Suitably the LNPs have a polydispersity of 0.4 or less, such as 0.3 or less.

In one embodiment the carrier is a lipid nanoparticle (LNP).

The hydrophilic portion of a lipid can be PEGylated (i.e. modified by covalent attachment of a polyethylene glycol). This modification can increase stability and prevent non-specific adsorption of the liposomes. For instance, lipids can be conjugated to PEG using techniques such as those disclosed in Heyes et al. (2005) J Controlled Release 107:276-87 and WO 2005/121348. Various lengths of PEG can be used e.g. between 0.5-8 kDa.

### Pharmaceutical Compositions and Formulations

The RNA-LNP is formulated to maintain percent encapsulation of the RNA as compared to a control, and preferably a pre-lyophilized control, as well as a size and PDI comparable to the control. The composition may also comprise one or more excipients including primary sugars, and optionally, one or more secondary sugars, amino acids, and plasticizers as well as a suitable buffer to improve and/or maintain percent encapsulation during freeze-drying.

Thus, the invention provides a pharmaceutical composition formulated for lyophilization of lipid nanoparticle encapsulated RNA (RNA-LNP), along with kits and vaccines. In aspects, the RNA comprises an mRNA encapsulated in a lipid nanoparticle (LNP). In aspects, the mRNA-LNP comprises a self-amplifying mRNA (SAM) encapsulated in a lipid nanoparticle (LNP) or SAM-LNP. In aspects, the mRNA-LNP comprises a mRNA encapsulated in a lipid nanoparticle (LNP) that is not self-amplifying (e.g., a non-replicating mRNA). The composition is designed to maintain the stability of the LNP encapsulated RNA during the lyophilization process and to allow storage under standard refrigeration temperatures or at room temperature. A novel lyophilization process is presented, for the compositions provided herein, for development of a thermostable composition (e.g., for a vaccine or drug product) to eliminate the need for cold chain storage by allowing storage at 5° C. up to room temperature, thus making stockpiling and distribution of said compositions feasible. However, when available and practical, the lyophilized composition can be stored at standard refrigeration temperatures above 0° C. and below room temperature which is usually considered to be 20, 21 or 22° C. For maximum storage stability, the lyophilized composition may be stored at temperatures just above 0° C. to up to 5° C., preferably just above 0° C. to up to 4° C., and more preferably 1° C. to 4° C. After lyophilization, the lyophilized composition will be stored for a period of time to allow for distribution to a site where the vaccine will be reconstituted and administered. Therefore, considering the fact that the vaccine may be need to be stockpiled for future use and that there may be some delays in distribution and administration, it may be necessary to store the vaccine for up to one year or longer, but the vaccine will preferably be stored for a shorter time such as 9 months, 6 months, 3 months or less.

As referenced herein, a primary sugar refers to a sugar present in the pharmaceutical composition in an amount of about 5% (w/v) or greater. Typically, the primary sugar may be present in an amount of 5-30% (w/v), about 5-20% (w/v), about 5-10% (w/v), or about 7.5% (w/v). In certain embodiments, the primary sugar is sucrose, preferably at 7.5% (w/v).

As referenced herein, a secondary sugar refers to a sugar present in the pharmaceutical composition in an amount from 0.1% to 5% (w/v) or about 2.5% (w/v). In certain embodiments, the secondary sugar is glucose, trehalose, maltose, or melezitose. A secondary sugar is in addition to the primary sugar, and in certain embodiments, is optional.

An amino acid refers to any of the naturally occurring amino acids or synthesized amino acids. In certain embodiments, the amino acid is arginine, methionine, histidine, lysine or alanine. In certain embodiments, the amino acid is present in an amount from 0.1% to 1.0% (w/v), about 0.1% to 1.0% (w/v) or about 0.5% (w/v).

As referenced herein, a plasticizer refers to a component that lowers the glass transition temperature (Tg), thereby increasing molecular mobility of the LNP. In aspects, plasticizers may be small molecules, such as glycerol or sorbitol or ethylene glycol, that help fill small open volumes arising from larger molecules. In certain embodiments, the plasticizer is present in an amount from about 0.1% to 1.0% (w/v) or about 1% (w/v).

Any suitable buffer may be used with the embodiments provided herein. Suitable buffers include but are not limited to Tris, histidine, phosphate, citrate, and HEPES buffer. Buffers may be at any suitable concentration including 5 mM-20 mM and may have any suitable pH ranging from 6 to 9.5.

Formulations with a Primary Sugar

In an embodiment, the pharmaceutical composition may comprise RNA-LNP (e.g., mRNA, SAM-LNP) formulated with about 5% (w/v) or greater of a primary sugar such as sucrose. For example, the primary sugar may be present in an amount of about 5-30% (w/v), about 5-20% (w/v), about 5-15% (w/v), about 5-10% (w/v), or about 7.5% (w/v). For example, the composition may comprise RNA-LNP formulated with sucrose in Tris buffer at a concentration ranging from 5-20 mM at physiological pH (e.g., 7.5-9 pH). In aspects, the formulation may comprise RNA-LNP formulated with 7.5% (w/v) sucrose in 20 mM Tris and 5 mM NaCl buffer at physiological pH (e.g., pH 8.0).

In another aspect, an optional plasticizer may be present in an amount of about 0.1-1% (w/v), and preferably about 0.5% (w/v).

In aspects, the composition may comprise any suitable buffer, including Tris, phosphate, citrate, HEPES and histidine buffer at any suitable concentration (e.g., 5 mM-20 mM) and suitable pH (e.g., 6-9.5).

In aspects, SAM-LNP may be at a concentration of about 60, 30, 10, 5, 2 and 1 μg/mL SAM-LNP, or a concentration equal to or less than each of these amounts. The LNP may comprise a cationic lipid, a zwitterionic lipid, and a cholesterol. The lipid nanoparticle may optionally comprise a PEGylated lipid. In some aspects, the lipid nanoparticle may comprise or consist of DSPC, DlinDMA, PEG-DMG and cholesterol. In certain embodiments, the lipid nanoparticle may comprise or consist of DSPC, RV39, PEG and cholesterol.

Formulations with a Primary Sugar, Secondary Sugar, Amino Acid and Plasticizer

In aspects, the composition may comprise RNA-LNP, a primary sugar, a secondary sugar (optional), an amino acid, and a plasticizer.

RNA-LNP, mRNA-LNP, and in particular SAM-LNP, may be present at a concentration of about 180 ug/mL or more, such as about 180-250 ug/mL, about 180-220 ug/mL, about 190-210 ug/mL, or preferably about 200 ug/mL. The LNP may comprise a cationic lipid, a zwitterionic lipid, and a cholesterol. The lipid nanoparticle may optionally comprise a PEGylated lipid. In some aspects, the lipid nanoparticle may comprise or consist of DSPC, DlinDMA, PEG-DMG and cholesterol. In certain embodiments, the lipid nanoparticle may comprise or consist of DSPC, RV39, PEG and cholesterol.

In aspects, the pharmaceutical composition may comprise RNA-LNP with:
1. a primary sugar such as sucrose;
2. optionally, a secondary sugar such as glucose, trehalose, maltose or melezitose;
3. one or more amino acids selected from the group consisting of arginine, methionine, histidine, lysine and alanine; and
4. a plasticizer such as glycerol or sorbitol.

The primary sugar may be present in an amount of 5-30% (w/v), about 5-20% (w/v), about 5-15% (w/v), about 5-10% (w/v), or about 7.5% (w/v). For example, the composition may comprise RNA-LNP formulated with sucrose in Tris buffer at a concentration ranging from 5-20 mM at a suitable pH (e.g., 7.5-9 pH). In aspects, the formulation may comprise RNA-LNP formulated with 7.5% (w/v) sucrose in 20 mM Tris and 5 mM NaCl buffer at physiological pH (e.g., 7.5-9 pH).

In certain embodiments, the pharmaceutical composition comprises a primary sugar (e.g., sucrose) at a concentration of about 5-10% (w/v) and preferably about 7.5% (w/v); and a secondary sugar (e.g., trehalose, glucose, maltose, or melezitose) at a concentration of about 0-5% (w/v) and preferably about 2.5% (w/v).

In aspects, the one or more amino acids are selected from the group consisting of arginine, lysine, histidine, methionine, alanine. The one or more amino acids may be present in an amount of about 0.1-1.0% (w/v) or about 0.1-1% (w/v), and preferably about 0.5% (w/v).

In aspects, the plasticizer is selected from the group consisting of sorbitol, glycerol, and ethylene glycol. In another aspect, the plasticizer may be present in an amount of about 0.1-1% (w/v), and preferably about 0.5% (w/v).

Formulations may include any suitable buffer including Tris (pH of 7.5-9.5), HEPES (pH of 7-9.0), phosphate (pH of 7-8.5), histidine (pH 6-7) or citrate buffer (pH of 6-7). In some aspects, suitable buffers include a range of 7.1-9.1, a pKa of 8.07 at room temperature, and have a percentage encapsulation up to ≤80% post lyophilization. Any suitable buffer concentration may be used (e.g., 5, 10 or 20 mM). In certain embodiments, the formulation may comprise 20 mM Tris and 5 mM NaCl buffer at physiological pH (e.g., pH 8.0).

In aspects, the primary sugar is sucrose, the amino acid is methionine, and the plasticizer is glycerol. In certain embodiments, sucrose is present in an amount of about 5-10% (w/v) or about 7.5% (w/v), methionine is present in an amount of about 0.1-1.0% or about 0.1-1.0% or about 0.25-0.75% (w/v), and glycerol is present in an amount of about 0.1-1% or about 0.25-0.75% (w/v).

In aspects, the composition comprises RNA-LNP (e.g., mRNA-LNP or SAM-LNP), sucrose, trehalose, methionine, and glycerol. For example, the composition comprises a primary sugar such as sucrose in an amount of about 7.5% (w/v), an amino acid such as methionine in an amount of about 0.5% (w/v), and a plasticizer such as glycerol in an amount of about 0.5% (w/v). As another example, sucrose is present in an amount of about 1-10% or about 5-10% (w/v), trehalose is present in an amount of about 0-5% (w/v), methionine is present in an amount of about 0.1-1.0% or about 0.1-1% or about 0.25-0.75% (w/v), and glycerol is present in an amount of about 0.1-1% or about 0.25-0.75% (w/v). In still other aspects, the composition comprises SAM-LNP, a primary sugar such as sucrose in an amount of about 5% (w/v), a secondary sugar such as trehalose in an amount of about 2.5% (w/v), an amino acid such as methionine in an amount of about 0.5% (w/v), and a plasticizer such as glycerol in an amount of about 0.5% (w/v).

Formulations may include any suitable buffer including Tris (pH of 7.5-9.5), HEPES (pH of 7-9.0), phosphate (pH of 7-8.5), histidine (pH 6-7) or citrate buffer (pH of 6-7). In some aspects, suitable buffers include a range of 7.1-9.1, a pKa of 8.07 at room temperature, and have a percentage encapsulation up to ≤80% post lyophilization. Any suitable buffer concentration may be used (e.g., 5, 10 or 20 mM of the buffer).

Formulations with a Primary Sugar and Histidine Buffer

In certain embodiments, the pharmaceutical composition may comprise RNA-LNP (e.g., wherein the RNA is mRNA, and the mRNA is a self-amplifying mRNA (SAM) or a non-replicating mRNA) formulated with about 5-10% sucrose in Histidine buffer. For example, the pharmaceutical composition may comprise RNA-LNP formulated about with 5-10% sucrose in 5 mM, 10 mM or 20 mM Histidine buffer at a pH of 6-7. In aspects, formulations may comprise mRNA (e.g., SAM-LNP DP), with sucrose (7.5% (w/v)), and 20 mM Histidine buffer at a pH from 6-7.

The pharmaceutical composition with sucrose and 20 mM histidine buffer may additionally and/or optionally comprise one or more of a secondary sugar, an amino acid, and a plasticizer. The secondary sugar may be selected from the group consisting of: glucose, trehalose, maltose or melezitose. The secondary sugar may be present in an amount of about 0-5% (w/v) or about 2.5% (w/v). The amino acid may be selected from the group consisting of: arginine, methionine, histidine, lysine and alanine. The amino acid may be present in an amount of about 0.1-1.0% (w/v) or 0.1-1.0% (w/v). The plasticizer may be selected from the group consisting of: glycerol or sorbitol or ethylene glycol. The plasticizer may be present in an amount of about 0.1-1% (w/v).

In certain embodiments, the primary sugar is sucrose, the amino acid is methionine, and the plasticizer is glycerol. For example, the primary sugar is sucrose 7.5% (w/v), the amino acid is methionine 1.0% (w/v), and the plasticizer is glycerol 1.0% (w/v).

### Combinations

It is contemplated herein that sucrose concentrations greater than or equal to 7.5%, in combination with one or more amino acids, buffers such as histidine, and in combination with the optimized lyophilization processes provided herein, will lead to further improvements in percent encapsulation, particle size, and PDI while retaining efficacy.

Pharmaceutical compositions provided herein (e.g., SAM-LNP about 200 ug/mL in 20 mM Tris, 5 mM NaCl and 7.5% sucrose) may be formulated with sterile water or with any suitable buffer. Buffers include but are not limited to citrate buffer with a pH of 6 to 7, histidine buffer with a pH of 6 to 7, phosphate buffer with a pH of 6.5 to 8.5, and HEPES buffer with a pH of 6.5 to 9. Buffer salts will typically be included in the 5-20 mM range. Pharmaceutical compositions of the invention may have a pH between 5.0 and 9.5, or between 6.0 and 9 or any other suitable range.

In other aspects, the pharmaceutical composition does not include dextran, PVP, Tween 20, or P-188. In other aspects, the pharmaceutical composition does not include 0.1% dextran, 1% PVP, 1% Tween 20, or 1% P188.

The compositions provided herein maintain and/or improve percent encapsulation comparable to a control formulation.

For the compositions provided herein, in certain embodiments, sucrose is present in an amount greater than or equal to 5% (w/v) or preferably 7.5% (w/v). Additionally plasticizers are present in an amount of about 0.5% (w/v).

RNA will be administered as a component in a pharmaceutical composition for immunising subjects against various diseases. These compositions will typically include a pharmaceutically acceptable carrier in addition to the RNA, often as part of a delivery system as described above. A thorough discussion of pharmaceutically acceptable carriers is available in Gennaro (2000) Remington: The Science and Practice of Pharmacy. 20th edition, ISBN: 0683306472.

A pharmaceutical composition of the invention may include one or more small molecule immunopotentiators. For example, the composition may include a TLR2 agonist (e.g. Pam3CSK4), a TLR4 agonist (e.g. an aminoalkyl glucosaminide phosphate, such as E6020), a TLR7 agonist (e.g. imiquimod), a TLR8 agonist (e.g. resiquimod) and/or a TLR9 agonist (e.g. IC31). Any such agonist ideally has a molecular weight of <2000 Da. Where an RNA is encapsulated, in some embodiments such agonist(s) are also encapsulated with the RNA, but in other embodiments they are unencapsulated. Where an RNA is adsorbed to a particle, in some embodiments such agonist(s) are also adsorbed with the RNA, but in other embodiments they are unadsorbed.

Compositions of the invention may include metal ion chelators. These can prolong RNA stability by removing ions which can accelerate phosphodiester hydrolysis. Thus a composition may include one or more of EDTA, EGTA, BAPTA, pentetic acid, etc. Such chelators are typically present at between 10-500 μM e.g. 0.1 mM. A citrate salt, such as sodium citrate, can also act as a chelator, while advantageously also providing buffering activity.

Pharmaceutical compositions of the invention may have an osmolality of between 200 mOsm/kg and 400 mOsm/kg, e.g. between 240-360 mOsm/kg, or between 290-310 mOsm/kg.

Pharmaceutical compositions of the invention may include one or more preservatives, such as thiomersal or 2-phenoxyethanol. Mercury-free compositions are preferred, and preservative-free vaccines can be prepared.

Pharmaceutical compositions of the invention are preferably sterile.

Pharmaceutical compositions of the invention are preferably non-pyrogenic e.g. containing <1 EU (endotoxin unit, a standard measure) per dose, and preferably <0.1 EU per dose.

Pharmaceutical compositions of the invention are preferably gluten free.

Pharmaceutical compositions of the invention may be prepared in unit dose form. In some embodiments a unit dose may have a volume of between 0.1-1.0 ml e.g. about 0.5 ml.

The compositions may be prepared as injectables, either as solutions or suspensions. Injectables for intramuscular administration are typical. Compositions may be lyophilized and reconstituted prior to administration.

Compositions comprise an immunologically effective amount of RNA, as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesize antibodies, the degree of protection desired, the formulation of the vaccine, the treat-ing doctor's assessment of the medical situation, and other rel-evant factors. It is expected that the amount will fall in a relatively broad range that can be determined through routine trials. The RNA content of compositions of the invention will generally be expressed in terms of the amount of RNA per dose. A preferred dose has ≤10 μg RNA, and expression can be seen at much lower levels e.g. ≤1 μg/dose, ≤100 ng/dose, ≤10 ng/dose, ≤1 ng/dose, etc.

The invention also provides a delivery device (e.g. syringe, etc.) containing a pharmaceutical composition of the invention. This device can be used to administer the composition to a vertebrate subject.

RNAs are not delivered in combination with ribosomes and so pharmaceutical compositions of the invention are ribosome-free.

### Lyophilization

Various lyophilization techniques are known in the art and can be used in accordance with the present invention for preparation of low-dose LNPs. In addition, a novel lyophilization process is presented, for the compositions provided herein, for development of a thermostable composition (e.g., for a vaccine or drug product) to eliminate the need for cold chain storage by allowing storage at 5° C. up to room temperature, thus making stockpiling and distribution of said compositions feasible.

The lyophilization process comprises freezing, primary drying, and secondary drying steps. For each process step, a cycle time, temperature, and pressure is selected along with a ramp time to achieve and maintain a desired temperature and pressure of the lyophilization chamber containing the pharmaceutical compositions for that process step.

In aspects, the lyophilization process may be used with the formulations provided herein, for example, a product containing liposome encapsulated mRNA, such as SAM-LNP or non-replicating mRNA.

The formulation is designed to preserve the percent encapsulation of the mRNA. Ideally, the formulation is designed to maintain a percent encapsulation of mRNA that is within 10%, 5%, 2% or better than a liquid control that does not undergo lyophilization. During the lyophilization process, samples may be obtained of the pharmaceutical composition, and may be characterized in terms of size, PDI and percent encapsulation as well as efficacy. Any suitable lyophilization device may be used for performing lyophilization of the compositions provided herein.

A sample loading step is performed to load the pharmaceutical composition into a lyophilization chamber. A first thermal equilibrium step is performed with a ramp rate of 1° C. per minute to reach a temperature of 5° C. for a duration 0.5 hours. Once completing the first thermal equilibrium step, a second thermal equilibrium step is performed. The second thermal equilibrium step is performed with a ramp rate of 1° C. per minute to reach a temperature of minus 5° C. for a duration of about 0.5 hours.

### Freezing

The composition, which is in a solution at room temperature, is subjected to primary freezing. Typically, primary freezing is performed at about minus 40° C., with a freezing rate ranging from about 0.1° C./min to about 1.0° C./min. Once reaching the desired freezing temperature, the lyophilization chamber may be held at this temperature for one hour or more.

### Primary Drying

After freezing, the frozen composition may undergo a primary drying step. Primary drying removes moisture from the frozen sample. Conditions for primary drying may include a temperature ranging from minus 25° C. to minus 35° C. (preferably at about minus 29 or minus 30° C.), held at the designated temperature for 25 or more hours at a pressure of about 55-60 mTorr. Ramp rates (freezing rates) during primary drying may range from 0.1° C./min up to 1.0° C./min, e.g., 0.5° C./min. In certain embodiments, the primary drying step may comprise a temperature of minus 29° C. for 27 hours at a vacuum pressure of 57 mTorr.

### Secondary Drying

After primary drying, the composition may undergo a secondary drying step. Secondary drying removes additional moisture from the product of primary drying. Conditions for secondary drying may include a temperature ranging from 0° C. to 25° C. or higher, held at the designated temperature for 8-48 hours or more at a pressure of about 55-60 mTorr. The ramp rate may be about 0.1° C./min.

In aspects, the secondary drying temperature may range from 5 to 40° C. and may include a drying duration time of 4 hours to 48 hours. In some aspects, the secondary drying temperature may be 5° C. for about 48 hours. In another aspect, the secondary drying temperature may be about 15° C. for about 12 hours. In another aspect, the secondary drying temperature may be about 25° C. for about 6 hours. In another aspect, the secondary drying temperature may be about 40° C. for about 4 hours. In aspects, the vacuum pressure during secondary drying is 60 mTorr and the ramp rate is about 0.1° C./min.

In certain embodiments, a secondary drying temperature of 5° C. for about 48 hours leads to about a 10% entrapment drop. A secondary drying temperature of about 15° C. for about 12 hours leads to about a 14% entrapment drop. As another example, a 25° C. secondary drying temperature for 6 hours leads to about a 17% entrapment drop. Accordingly, a temperature range of 5-15° C. for 12-48 hours minimizes the drop in percentage entrapment.

It was surprisingly observed that the percent entrapment initially decreased during secondary drying but returned toward its initial value by the end of secondary drying. It was also observed that the particle size initially increased during secondary drying but returned towards its initial value by the end of secondary drying. Additionally, it was observed that potency was initially reduced during secondary drying but returned towards its initial value by the end of secondary drying.

### Administration

The invention involves administration of a composition comprising an RNA-LNP molecule, wherein the composition has been lyophilized and reconstituted, to a delivery site in a vertebrate. The delivery site will usually be muscle tissue, such as skeletal muscle. Alternatives to intramuscular administration include, but are not limited to: intradermal, intranasal, intraocular, subcutaneous, intraperitoneal, intravenous, interstitial, buccal, transdermal, or sublingual administration. Intradermal and intramuscular administration are two preferred routes.

Administration can be achieved in various ways. For instance, injection via a needle (e.g. a hypodermic needle) can be used, particularly for intramuscular, subcutaneous, intraocular, intraperitoneal or intravenous administration. Needle-free injection can be used as an alternative.

Intramuscular injection is the preferred way of administering RNA according to the invention. Injection into the upper arm, deltoid or thigh muscle (e.g. anterolateral thigh) is typical.

The delivery site includes non-immune cells, such as muscle cells (which may be multinucleated and may be arranged into fascicles) and/or fibroblasts. RNA enters the cytoplasm of these cells after (or while) being administered to the delivery site. Entry can be via endocytosis e.g. across the sarcolemma of a muscle cell, or across the cell membrane of a fibroblast. After RNA entry (and escape from the endosome), it can bind to RNA helicases such as RIG-I (RLR-1), MDA5 (RLR-2) and/or LGP2 (RLR-3). This binding can initiate RLR-mediated signalling, thereby triggering innate immune pathways which enhance the immunogenic effect of the delivered RNA. Even if the delivered RNA is single-stranded, it can form double-stranded RNA either during replication or due to its secondary structure, which means that the RNA can also initiate PKR-mediated signaling, again leading to the triggering of innate immune pathways. Both RLR-mediated and PKR-mediated signaling can lead to secretion of type I interferons (e.g. interferon α and/or β) by the non-immune cells. The non-immune cells may undergo apoptosis after transfection.

The delivery site also includes immune cells, such as macrophages (e.g. bone marrow derived macrophages), dendritic cells (e.g. bone marrow derived plasmacytoid dendritic cells and/or bone marrow derived myeloid dendritic cells), monocytes (e.g. human peripheral blood monocytes), etc. These immune cells can be present at the delivery site at the time of administration, but will usually infiltrate the delivery site after administration. For example, the tissue damage caused by invasive administration (e.g. caused at the delivery site by a needle) can cause immune cells to infiltrate the damaged area. These infiltrating cells will encounter the RNA which is now at the delivery site and RNA can enter the cytoplasm of these immune cells e.g. via endocytosis. After RNA entry the response of these immune cells includes secretion of type I interferons and/or pro-inflammatory cytokines. The RNA can cause this effect via pattern-recognition receptors, such as toll-like receptors (e.g. TLR7), intracellular helicases (e.g. RIG-I), and PKR (dsRNA-dependent protein kinase). The RNA may or may not be translated by the immune cells, and so the immune cells may or may not express the immunogen. If the immunogen is expressed by the immune cell then it may be presented by the immune cell's MHC-I and/or MHC-II. If the immunogen is not expressed by the immune cell then it may instead be captured by the immune cell from other cells (e.g. non-immune cells) which had taken up RNA and expressed the immunogen, and the immunogen can thus be presented by the immune cell's MHC-II and/or MHC-I. Antigen presentation will generally occur in draining lymph nodes after immune cells have migrated away from the delivery site.

The infiltration of immune cells at a delivery site can be observed directly e.g. by using labelled immune cells and then either in vivo imaging techniques or biopsy sampling. The ability of RNA to enter the immune cells and the non-immune cells can be detected by sequence-specific detection techniques performed in situ or on an excised sample.

### Subjects

The present invention is generally intended for mammalian subjects, in particular human subjects. The subject may be a wild or domesticated animal. Mammalian subjects include for example cats, dogs, pigs, sheep, horses or cattle. In one embodiment the invention, the subject is human.

The subject to be treated using the method of the invention may be of any age.

In one embodiment the subject is a human infant (up to 12 months of age). In another embodiment the subject is a human child between the ages of 6 months up to 11 years, 5 years up to 11 years, 12 years up to 16 or 17 years. In one embodiment the subject is a human child (less than 18 years of age). In one embodiment the subject is an adult human (aged 18-59). In one embodiment the subject is an older human (aged 60 or greater).

Doses administered to younger children, such as less than 12 years of age, may be reduced relative to an equivalent adult dose.

The methods of the invention are suitably intended for prophylaxis of infectious diseases, i.e. for administration to a subject which is not infected with a pathogen. In other embodiments the methods of the invention may be intended for treatment, e.g. for the treatment of infectious diseases, i.e. for administration to a subject which is infected with a pathogen.

Compositions of the invention may be co-administered with other formulations such as adjuvants. Consequently, it will be appreciated that a range of formulation possibilities exist. A reasonable balance is desirable between practical considerations such as: manufacture, storage and distribution of the mRNA. The carrier-formulated mRNA and adjuvant are desirably administered to locations with sufficient spatial proximity such that the adjuvant effect is adequately maintained. For example, spatial proximity is sufficient to maintain at least 50%, especially at least 75% and in particular at least 90% of the adjuvant effect seen with administration to the same location. The adjuvant effect seen with administration to the same location is defined as the level of increase observed as a result of administration of carrier-formulated mRNA and squalene emulsion adjuvant to the same location compared with administration of carrier-formulated mRNA alone. The carrier-formulated mRNA and adjuvant are desirably administered to a location draining to the same lymph node, such as to the same limb, in particular to the same muscle.

### Adjuvant

Suitably carrier-formulated reconstituted mRNA and adjuvant are administered intramuscularly to the same muscle. In certain embodiments, the carrier-formulated mRNA and adjuvant are administered to the same location.

The spatial separation of administration locations may be at least 5 mm, such as at least 1 cm. The spatial separation of administration locations may be less than 10 cm, such as less than 5 cm apart.

When administered as separate formulations, the carrier-formulated mRNA and adjuvant are desirably administered with sufficient temporal proximity such that the adjuvant effect is adequately maintained. For example, temporal proximity is sufficient to maintain at least 50%, especially at least 75% and in particular at least 90% of the adjuvant effect seen with administration at the same time. The adjuvant effect seen with administration at the same time is defined as the level of increase observed as a result of administration of carrier-formulated mRNA and adjuvant at (essentially) the same time compared with administration of carrier-formulated mRNA without adjuvant.

When administered as separate formulations, carrier-formulated mRNA and adjuvant may be administered within 12 hours. Suitably the carrier-formulated mRNA and adjuvant are administered within 6 hours, especially within 2 hours, in particular within 1 hour, such as within 30 minutes and especially within 15 minutes (e.g. within 5 minutes).

When administered as separate formulations, carrier-formulated mRNA and adjuvant may be administered within 84 hours, such as within 60 hours, especially within 36 hours, in particular within 24 hours. In one embodiment the carrier-formulated mRNA and adjuvant are administered within 12 to 36 hours. In another embodiment the carrier-formulated mRNA and adjuvant are administered within 36 to 84 hours.

The delay between administration of the carrier-formulated mRNA and adjuvant may be at least 5 seconds, such as 10 seconds, and in particular at least 30 seconds.

When administered as separate formulations, if the carrier-formulated mRNA and adjuvant are administered with a delay, the carrier-formulated mRNA may be administered first and the adjuvant administered second. Alternatively, the adjuvant is administered first and the carrier-formulated mRNA is administered second. Appropriate temporal proximity may depend on the order of administration.

In addition to co-formulated or separately formulated presentations of carrier-formulated mRNA and adjuvant for direct administration, the carrier-formulated mRNA and adjuvant may initially be provided in various forms which facilitate manufacture, storage and distribution. For example, certain components may have limited stability in liquid form, certain components may not be amendable to drying, certain components may be incompatible when mixed (either on a short- or long-term basis). Independent of whether carrier-formulated mRNA and are co-formulated at administration, they may be provided in separate containers the contents of at least some of which are subsequently combined. The skilled person will appreciate that many possibilities exist, although it is generally desirable to have a limited number of containers and limited number of required steps to prepare the final co-formulation or separate formulations for administration.

Carrier-formulated mRNA (e.g., liposomal encapsulated mRNA) may be provided in liquid or dry (e.g. lyophilized) form. The preferred form will depend on factors such as the precise nature of the carrier-formulated mRNA, e.g. if the carrier-formulated mRNA is amenable to drying, or other components which may be present.

The adjuvant may be provided in liquid or dry form. The preferred form will depend on the precise nature of adjuvant, e.g. if capable of self-emulsification, and any other components present.

The invention provides a composition comprising carrier-formulated mRNA encoding an antigen and an adjuvant. Typically carrier-formulated mRNA encoding an antigen and adjuvant are provided as a liquid co-formulation. A liquid co-formulation enables convenient administration at the point of use.

In other embodiments the carrier-formulated mRNA encoding an antigen and adjuvant are provided as a dry co-formulation, the dry co-formulation being reconstituted prior to administration. A dry co-formulation, where the components of the formulation are amendable to such presentation, may improve stability and thereby facilitate longer storage.

The carrier-formulated mRNA encoding an antigen and adjuvant may be provided in separate containers. The invention therefore provides carrier-formulated mRNA encoding an antigen for use with an adjuvant. Also provided is an adjuvant for use with carrier-formulated mRNA encoding an antigen. Further provided is a kit comprising:
a first container comprising carrier-formulated mRNA encoding an antigen; and
a second container comprising an adjuvant.

The carrier-formulated mRNA encoding an antigen may be in liquid form and the adjuvant may be in liquid form. In such cases the contents of the first and second containers may be intended for combination to provide a co-formulation for administration. Alternatively, the contents of each container may be intended for separate administration as the first and second formulations.

The carrier-formulated mRNA encoding an antigen may be in dry form and the adjuvant may be in liquid form. In such cases the contents of the first and second containers may be intended for combination to provide a co-formulation for administration. Alternatively, the carrier-formulated mRNA encoding an antigen may be intended to be reconstituted prior to the contents of each container being used for separate administration as the first and second formulations.

The adjuvant may be in dry form and the carrier-formulated mRNA encoding an antigen may be in liquid form. In such cases the contents of the first and second containers may be intended for combination to provide a co-formulation for administration. Alternatively, the adjuvant may be intended to be reconstituted prior to the contents of each container being used for separate administration as the first and second formulations.

The carrier-formulated mRNA may be in dry form (lyophilized) and the adjuvant may be in dry form. In such cases the contents of the first and second containers may be intended for reconstitution and combination to provide a co-formulation for administration. Reconstitution may occur separately before combination, or the contents of one container may be reconstituted and then used to reconstitute the contents of the other container. Alternatively, the contents of the first and second containers may be intended for reconstitution prior to the contents of each container being used for separate administration as the first and second formulations. In other cases, the carrier-formulated mRNA may be in dry form, and may be reconstituted with the adjuvant in liquid form.

The precise composition of liquid used for reconstitution will depend on both the contents of a container being reconstituted and the subsequent use of the reconstituted contents e.g. if they are intended for administration directly or may be combined with other components prior to administration. A composition (such as those containing carrier-formulated mRNA encoding an antigen or adjuvant) intended for combination with other compositions prior to administration need not itself have a physiologically acceptable pH or a physiologically acceptable tonicity; a formulation intended for administration should have a physiologically acceptable pH and should have a physiologically acceptable osmolality.

The pH of a liquid preparation is adjusted in view of the components of the composition and necessary suitability for administration to the human subject. The pH of a formulation is generally at least 4, especially at least 5, in particular at least 5.5 such as at least 6. The pH of a formulation is generally 9 or less, especially 8.5 or less, in particular 8 or less, such as 7.5 or less. The pH of a formulation may be 4 to 9, especially 5 to 8.5, in particular 5.5 to 8, such as 6.5 to 7.4 (e.g. 6.5 to 7.1).

For parenteral administration, solutions should have a physiologically acceptable osmolality to avoid excessive cell distortion or lysis. A physiologically acceptable osmolality will generally mean that solutions will have an osmolality which is approximately isotonic or mildly hypertonic. Suitably the formulations for administration will have an osmolality of 250 to 750 mOsm/kg, especially 250 to 550 mOsm/kg, in particular 270 to 500 mOsm/kg, such as 270 to 400 mOsm/kg. Osmolality may be measured according to techniques known in the art, such as by the use of a commercially available osmometer, for example the Advanced@Model 2020 available from Advanced Instruments Inc. (USA).

Liquids used for reconstitution will typically be substantially aqueous, such as water for injection, phosphate buffered saline and the like. As mentioned above, the requirement for buffer and/or tonicity modifying agents will depend on both the contents of the container being reconstituted and the subsequent use of the reconstituted contents. Buffers may be selected from acetate, citrate, histidine, maleate, phosphate, succinate, tartrate and TRIS. The buffer may be a phosphate buffer such as Na/Na2PO4, Na/K2PO4 or K/K2PO4.

Suitably, the formulations used in the present invention have a dose volume of between 0.05 ml and 1 ml, such as between 0.1 and 0.6 ml, in particular a dose volume of 0.45 to 0.55 ml, such as 0.5 ml. The volumes of the compositions used may depend on the subject, delivery route and location, with smaller doses being given by the intradermal route or if both the carrier-formulated mRNA and adjuvant are delivered to the same location. A typical human dose for administration through routes such as intramuscular, is in the region of 200 ul to 750 ml, such as 400 to 600 ul, in particular about 500 ul, such as 500 ul.

If two liquids are intended to be combined, for example for co-formulation if the carrier-formulated mRNA is in liquid form and the adjuvant is in liquid form, the volume of each liquid may be the same or different. Volumes for combination will typically be in the range of 10:1 to 1:10, such as 2:1 to 1:2. Suitably the volume of each liquid will be substantially the same, such as the same. For example a 250 ul volume of carrier-formulated mRNA in liquid form may be combined with a 250 ul volume adjuvant in liquid form to provide a co-formulation dose with a 500 ul volume, each of the carrier-formulated mRNA and adjuvant being diluted 2-fold during the combination.

It is common where liquids are to be transferred between containers, such as from a vial to a syringe, to provide ‘an overage’ which ensures that the full volume required can be conveniently transferred. The level of overage required will depend on the circumstances but excessive overage should be avoided to reduce wastage and insufficient overage may cause practical difficulties. Overages may be of the order of 20 to 100 ul per dose, such as 30 ul or 50 ul. For example, a typical 10 dose container of doubly concentrated squalene emulsion adjuvant (250 ul per dose) may contain around 2.85 to 3.25 ml of squalene emulsion adjuvant.

Carrier-formulated mRNA and adjuvant in liquid form may be provided in the form of a multichamber syringe. The use of multi-chamber syringes provides a convenient method for the separate sequential administration of the carrier-formulated mRNA and adjuvant. Multi-chamber syringes may be configured to provide concurrent but separate delivery of the carrier-formulated mRNA and adjuvant, or they may be configured to provide sequential delivery (in either order).

In other configurations of multichambered syringes, the carrier-formulated mRNA may be provided in dry form (e.g., freeze-dried) in one chamber and reconstituted by the adjuvant contained in the other chamber before administration.

Examples of multi-chamber syringes may be found in disclosures such as WO 2016/172396, although a range of other configurations are possible.

Formulations are preferably sterile.

Approaches for establishing strong and lasting immunity often include repeated immunisation, i.e. boosting an immune response by administration of one or more further doses. Such further administrations may be performed with the same immunogenic compositions (homologous boosting) or with different immunogenic compositions (heterologous boosting). The present invention may be applied as part of a homologous or heterologous prime/boost regimen, as either the priming or a/the boosting immunisation.

Administration of the carrier-formulated mRNA and adjuvant may therefore be part of a multi-dose administration regime. For example, the carrier-formulated mRNA and adjuvant may be provided as a priming dose in a multidose regime, especially a two- or three-dose regime, in particular a two-dose regime. The carrier-formulated mRNA and adjuvant may be provided as a boosting dose in a multidose regime, especially a two- or three-dose regime, such as a two-dose regime.

Priming and boosting doses may be homologous or heterologous. Consequently, the carrier-formulated mRNA and adjuvant may be provided as a priming dose and boosting dose(s) in a homologous multidose regime, especially a two- or three-dose regime, in particular a two-dose regime. Alternatively, the carrier-formulated mRNA and adjuvant may be provided as a priming dose or boosting dose in a heterologous multidose regime, especially a two- or three-dose regime, in particular a two-dose regime, and the boosting dose(s) may be different (e.g. a different carrier-formulated mRNA; or an alternative antigen presentation such as protein or virally vectored antigen—with or without adjuvant).

The time between doses may be two weeks to six months, such as three weeks to three months. In aspects, the time between doses is at least 21 days, 28 days, 42 days, 45 days, or 60 days. In aspects, the time between doses is at least 8 weeks, 10 weeks, 12 weeks, 14 weeks, or 16 weeks. Periodic longer-term booster doses may also be provided, such as every 2 to 10 years.

The adjuvant may be administered to a subject separately from carrier-formulated mRNA, or the adjuvant may be combined, either during manufacturing or extemporaneously, with carrier-formulated mRNA to provide an immunogenic composition for combined administration.

### Methods of Treatment and Medical Uses

RNA delivery according to the invention is for eliciting an immune response in vivo against an immunogen of interest. The immune response is preferably protective and preferably involves antibodies and/or cell-mediated immunity. The method may raise a booster response.

By raising an immune response the vertebrate can be protected against various diseases and/or infections e.g. against bacterial and/or viral diseases as discussed above. RNA-containing compositions are immunogenic, and are more preferably vaccine compositions. Vaccines according to the invention may either be prophylactic (i.e. to prevent infection) or therapeutic (i.e. to treat infection), but will typically be prophylactic.

The vertebrate is preferably a mammal, such as a human or a large veterinary mammal (e.g. horses, cattle, deer, goats, pigs). Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc.

Vaccines prepared according to the invention may be used to treat both children and adults. Thus a human patient may be less than 1 year old, less than 5 years old, 1-5 years old, 5-15 years old, 15-55 years old, or at least 55 years old. Preferred patients for receiving the vaccines are the elderly (e.g. >50 years old, >60 years old, and preferably >65 years), the young (e.g. <5 years old), hospitalised patients, healthcare workers, armed service and military personnel, pregnant women, the chronically ill, or immunodeficient patients. The vaccines are not suitable solely for these groups, however, and may be used more generally in a population.

Compositions of the invention will generally be administered directly to a patient. Direct delivery may be accomplished by parenteral injection (e.g. subcutaneously, intraperitoneally, intravenously, intramuscularly, or to the interstitial space of a tissue; unlike WO 02/02606, intraglossal injection is not typically used with the present invention), or mucosally, such as by rectal, oral (e.g. tablet, spray), vaginal, topical, transdermal or transcutaneous, intranasal, ocular, aural, pulmonary or other mucosal administration. Injection may be via a needle (e.g. a hypodermic needle), but needle-free injection may alternatively be used. A typical intramuscular dose is 0.5 ml.

The invention may be used to elicit systemic and/or mucosal immunity, preferably to elicit an enhanced systemic and/or mucosal immunity.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.). In one embodiment, multiple doses may be administered approximately 6 weeks, 10 weeks and 14 weeks after birth, e.g. at an age of 6 weeks, 10 weeks and 14 weeks, as often used in the World Health Organisation's Expanded Program on Immunisation (“EPI”). In an alternative embodiment, two primary doses are administered about two months apart, e.g. about 7, 8 or 9 weeks apart, followed by one or more booster doses about 6 months to 1 year after the second primary dose, e.g. about 6, 8, 10 or 12 months after the second primary dose. In a further embodiment, three primary doses are administered about two months apart, e.g. about 7, 8 or 9 weeks apart, followed by one or more booster doses about 6 months to 1 year after the third primary dose, e.g. about 6, 8, 10, or 12 months after the third primary dose.

Thus, in some embodiments, uses of the compositions, kits, immunogenic compositions, vaccines, or the reconstituted compositions in the manufacture of a medicament for treating a subject in need thereof are provided. In some embodiments, uses of the compositions, kits, immunogenic compositions, vaccines, or the reconstituted compositions in the manufacture of a medicament for prophylaxis in a subject in need thereof are provided.

Methods for eliciting an immune response in a subject in need thereof comprising administering the compositions, kits, vaccines, or the reconstituted compositions to the subject are provide. In some embodiments of these methods, the subject is a human subject.

In some embodiments are provided the compositions, kits, vaccines, or the reconstituted compositions for use in medicine.

### General Embodiments

In some embodiments of the invention, the RNA includes no modified nucleotides (see above). In other embodiments the RNA can optionally include at least one modified nucleotide.

The practice of the present invention will employ, unless otherwise indicated, conventional methods of chemistry, biochemistry, molecular biology, immunology and pharmacology, within the skill of the art. Such techniques are explained fully in the literature. See, e.g., Methods In Enzymology (S. Colowick and N. Kaplan, eds., Academic Press, Inc.); Handbook of Experimental Immunology, Vols. I-IV (D. M. Weir and C. C. Blackwell, eds, 1986, Blackwell Scientific Publications); Sambrook et al. (2001) Molecular Cloning: A Laboratory Manual, 3rd edition (Cold Spring Harbor Laboratory Press); Handbook of Surface and Colloidal Chemistry (Birdi, K. S. ed., CRC Press, 1997); Ausubel et al. (eds) (2002) Short protocols in molecular biology, 5th edition (Current Protocols); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); etc.

References to charge, to cations, to anions, to zwitterions, etc., are taken at pH 7.

TLR3 is the Toll-like receptor 3. It is a single membrane-spanning receptor which plays a key role in the innate immune system. Known TLR3 agonists include poly(I:C). “TLR3” is the approved HGNC name for the gene encoding this receptor, and its unique HGNC ID is HGNC:11849. The RefSeq sequence for the human TLR3 gene is GI:2459625.

TLR7 is the Toll-like receptor 7. It is a single membrane-spanning receptor which plays a key role in the innate immune system. Known TLR7 agonists include e.g. imiquimod. “TLR7” is the approved HGNC name for the gene encoding this receptor, and its unique HGNC ID is HGNC:15631. The RefSeq sequence for the human TLR7 gene is GI:67944638.

TLR8 is the Toll-like receptor 8. It is a single membrane-spanning receptor which plays a key role in the innate immune system. Known TLR8 agonists include e.g. resiquimod. “TLR8” is the approved HGNC name for the gene encoding this receptor, and its unique HGNC ID is HGNC:15632. The RefSeq sequence for the human TLR8 gene is GI:20302165.

The RIG-I-like receptor (“RLR”) family includes various RNA helicases which play key roles in the innate immune system (see Yoneyama & Fujita (2007) Cytokine & Growth Factor Reviews 18:545-51). RLR-1 (also known as RIG-I or retinoic acid inducible gene I) has two caspase recruitment domains near its N-terminus. The approved HGNC name for the gene encoding the RLR-1 helicase is “DDX58” (for DEAD (Asp-Glu-Ala-Asp) box polypeptide 58) and the unique HGNC ID is HGNC:19102. The RefSeq sequence for the human RLR-1 gene is GI:77732514. RLR-2 (also known as MDA5 or melanoma differentiation-associated gene 5) also has two caspase recruitment domains near its N-terminus. The approved HGNC name for the gene encoding the RLR-2 helicase is “IFIH1” (for interferon induced with helicase C domain 1) and the unique HGNC ID is HGNC:18873. The RefSeq sequence for the human RLR-2 gene is GI: 27886567. RLR-3 (also known as LGP2 or laboratory of genetics and physiology 2) has no caspase recruitment domains. The approved HGNC name for the gene encoding the RLR-3 helicase is “DHX58” (for DEXH (Asp-Glu-X-His) box polypeptide 58) and the unique HGNC ID is HGNC:29517. The RefSeq sequence for the human RLR-3 gene is GI:149408121.

PKR is a double-stranded RNA-dependent protein kinase. It plays a key role in the innate immune system. “EIF2AK2” (for eukaryotic translation initiation factor 2-alpha kinase 2) is the approved HGNC name for the gene encoding this enzyme, and its unique HGNC ID is HGNC:9437. The RefSeq sequence for the human PKR gene is GI:208431825.

### Making the Lyophilized Composition

The lyophilized composition can be made by any technique that produces a desired amount of SAM-LNPs or mRNA-LNPs- and a desired amount of empty LNPs. In addition, it is desirable that most of the mRNA is contained in LNPs, and preferably all or substantially all of the mRNA or SAM is contained in LNPs, as discussed above. It is convenient to initially make an aqueous buffered solution containing both mRNA-LNPs or SAM-LNPs and empty LNPs. This aqueous buffered solution is then lyophilized by techniques known in the art or as described in the Examples below until water has been substantially or completely removed from the composition and a lyophilized product is formed. It is understood that small amounts of water may remain in the composition, either in the core (inside) of the LNPs and/or on the exterior of the LNPs. The lyophilized composition is then stored at a convenient temperature and for a time necessary for distribution and administration to patients.

### Making RNA Containing Lipid Nanoparticles

The RNA-LNPs, mRNA-LNPs and SAM-LNPs can be made by variety of different techniques known in the art. A specific technique is described in the Examples below. These RNA-LNPs, mRNA-LNPs or SAM-LNPs can be stored until ready for mixing with empty LNPs in either liquid, frozen or lyophilized form.

### Making Empty Lipid Nanoparticles

Techniques for making empty LNPs are also known in the art. Briefly, these techniques can be essentially the same as techniques used to make RNA-LNPs, mRNA-LNPs or SAM-LNPs except that there is no RNA, mRNA and/or SAM inside the LNPs. Because the empty LNPs do not contain mRNA therein, they may have some physical properties that are different from the corresponding RNA-LNPs, mRNA-LNPs or SAM-LNPs. For example, the empty LNPs may be slightly smaller than the RNA-LNPs, mRNA-LNPs or SAM-LNPs. Thus, the average volume of the empty LNPs may be up to 20%, 15%, 10%, 8%, 5%, 3%, 2% or 1% smaller than the RNA-LNPs, mRNA-LNPs or SAM-LNPs. In addition, since the empty LNPs do not contain RNA, mRNA or SAM, it may be possible to make the empty LNPs from a lipid composition that is different from the lipid composition of the RNA-LNPs, mRNA-LNPs or SAM-LNPs. For example, the amount (relative proportion) of the cationic lipid in the empty LNP can be reduced with respect to the amount of cationic lipid in the RNA-LNPs, mRNA-LNPs or SAM-LNPs.

Combining the RNA-LNPs with the Empty LNPs

Empty LNP and mRNA-LNP (referred to interchangeably in this section hereafter as SAM-LNP) compositions can be prepared (and optionally lyophilized) separately from each other and combined or mixed at a desired ratio. Typically, at least one of the empty LNP composition or SAM-LNP composition will be in the form of a liquid at the time of combining or mixing; however, both the empty LNPs and the SAM-LNPs can be in the form of a lyophilized composition and these two lyophilized compositions can be mixed with a suitable liquid (such as a saline solution, water for injection and/or aqueous buffer) to form a mixed composition that it ready for lyophilization. It may be convenient to prepare an empty LNP composition and store it in a frozen form. Prior to mixing with the SAM-LNP composition, the empty LNP composition can be thawed. The thawed empty LNP composition can then be mixed with a SAM-LNP composition. This technique was used in the examples to prepare mixed compositions of differing concentrations of empty LNPs and SAM-LNPs by serial dilution with a buffer. However, in commercial manufacturing, a different process may be more convenient or desirable.

The SAM-LNPs and the empty LNPs can be mixed to provide the desired amount of empty LNPs. Based on the experiments reported herein, it has been surprisingly discovered that even a small amount of empty LNPs can stabilize or improve one or more properties of a lyophilized composition that does not contain empty LNPs. For example, for low-dose vaccines, it is difficult to make a SAM-LNP vaccine that has the same properties before lyophilization and after reconstitution. Thus, an effective amount of empty LNP can, in one embodiment, be an amount effective to reduce changes between properties of a vaccine composition pre-lyophilization and post-reconstitution, the post-reconstitution vaccine composition (which may or may not be further diluted) being ready for administration to a patient. Thus, these empty LNPs can also allow the lyophilized composition and a reconstituted composition made therefrom to retain similar properties, such as size and lipid composition. The amount of empty LNPs contained in the mixed composite on (containing mRNA-LNPs such as SAM-LNPs and empty LNPs) is preferably only an amount necessary to achieve a stabilizing effect. Typically, the amount of empty LNPs (measured in terms of the total weight of lipid in dry LNPs) is up to 25%, less than 25%, less than 20%, less than 15%, less than 10%, less than 5%, less than 4%, less than 3%, less than 2%, less than 1% or less than 0.5%, based on the total amount of lipid in SAM-LNPs and lipid in empty LNPs (measured in terms of the total dry weight of lipid in empty LNPs and the total dry weight of lipid in SAM-LNPs). The amounts of empty LNPs that can be present in accordance with the present invention includes amounts less than or equal to the amounts in the Examples in this application and also includes ranges, the endpoints of which correspond to amounts including and between any two amounts described in any two of the Examples of the invention in this application. Thus, in some embodiments, methods for manufacturing lyophilized compositions, immunogenic compositions, and/or vaccines comprising mRNA-LNPs, such as SAM-LNPs, and empty LNPs are provided comprising the steps of placing the composition into a sterile container; subjecting the composition to a drying step; and sealing the container. In some embodiments, the methods involve more than one drying steps.

### The Pre-Lyophilized Composition

The composition to be lyophilized (containing both RNA-LNPs, mRNA-LNPs or SAM-LNPs and excipients and empty LNPs) may be an aqueous buffered composition that is suitable for injection, but that is intended to be lyophilized to provide a stable formulation that can be stored and transported at the temperatures described herein. In this embodiment, the pre-lyophilized composition will have a chemical composition and physical properties that closely match the composition and properties of the reconstituted composition, as described below.

The amount of encapsulated mRNA and the percentage of empty LNPs are desirably about the same before lyophilization, optionally after lyophilization, and after reconstitution. The percentage of mRNA that is ideally encapsulated in LNPs is described in other parts of this application, but the percentage is typically is greater than 95%, 96%, 97%, 98%, 99% or more and as close to 100% as possible.

The pre-lyophilized composition may contain an effective amount of empty LNPs to stabilize the lyophilized composition and to also allow for efficient and effective reconstitution, as described further herein. Thus, the empty LNPs (which do not contain mRNA therein) can be characterized as a type of excipient that improves the physical properties of the composition. The amount of empty LNPs. The concentration of mRNA in the pre-lyophilized composition may be on the order of less than 60 μg/mL, wherein the mRNA is present in an effective amount upon reconstitution.

### Lyophilization

The lyophilized composition can be made by techniques known in the art and also as described in the present application.

### The Lyophilized Composition

The relative amounts of the RNA-LNPs, mRNA-LNPs or SAM-LNPs and the empty LNPs are typically the same as the relative amounts in the pre-lyophilized composition as described immediately above. The lyophilized composition will contain the RNA-LNPs, mRNA-LNPs or SAM-LNPs and the empty LNPs as well as any other non-liquid components of the liquid or aqueous composition that are not removed during lyophilization. The lyophilized composition will be in the form of a loose dry powder or a formed dry cake or a combination of the two. The dry powder or cake will have a certain volume that may be increased, may remain the same or may be decreased during reconstitution after enough liquid for reconstitution is added. Ideally, the dry powder or dry cake or combination of the two will be easily dispersed in the water for reconstitution with the aid of agitation of a degree typical in the pharmaceutical arts for reconstituting lyophilized vaccines at the facility where injections are administered prior to injection.

### Reconstituting the Lyophilized Composition

As discussed above, the vaccine can be part of a kit that contains the lyophilized low-dose composition of the present invention in one container and a liquid to be used for reconstitution in another container. The liquid for reconstitution can be sterile water for injection if the lyophilized composition contains all the necessary buffer components, adjuvants and/or excipients in dry form. In such a situation, after reconstitution with sterile water for injection, the resulting aqueous formulation will have acceptable properties for injection.

In one embodiment, an aqueous solution such as sterile water for injection is introduced into the container (such as a sterile vial) housing the lyophilized low-dose composition in an amount sufficient to provide the desired concentration of the various components of the in the injectable solution. The container will typically have enough of the lyophilized composition for multiple low doses, such as 2-10 doses or 3-8 doses or 4-8 doses or 5-6 doses. In this situation, the container will contain at least enough vaccine composition for the corresponding number of low doses, and usually some extra volume. After the aqueous solution is introduced into the vial, the vial is shaken and inspected for expected and desired visual properties, such as degree of transparency, color, etc. The injectable solution is then ready for being withdrawn from the vial by techniques known in the art, such as by inserting a syringe needle into the vial and withdrawing an appropriate amount of injectable solution into the syringe. Although sterile water for injection may be a suitable liquid for reconstitution, other aqueous solutions such as buffers containing other additive, excipients and/or adjuvants may be included in the liquid for reconstitution if these other additives, excipients and/or adjuvants are not present, or are not present in sufficient quantities, in the lyophilized composition.

Reconstitution can take place at any suitable temperature but will typically take place at room temperature.

When the vaccine is provided as a kit, the kit will typically include 2 or more containers containing different components that need to be mixed prior to injection into the subject being vaccinated. The two or more containers will include, for example, one container that contains the lyophilized low-dose composition and one container that contains the liquid for reconstitution. Alternatively, the kit may include one container that contains the lyophilized low-dose composition and a syringe of suitable size (packaged in a sterile package) and optionally another container that contains the liquid for reconstitution. Instructions for reconstitution at the clinic site and for administration of the reconstituted vaccine composition to the patient will be included in, on or associated with the various components of the kit.

### Definitions and Meaning of Terms

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

“About” or “approximately”, when used to modify a numeric value, means a number that is not statistically different from the referenced numeric value and, when the numeric value relates to the amount of a composition component, means a number not more than 10% below or above the numeric value (not more than 10% below or above the endpoint values if the numeric value is a range). As an example, a composition comprising “about 25 μg” of component A means the composition comprises “22.5-27.5 μg” of component A (10% of 25 is 2.5, so 10% below 25 is 22.5 and 10% above 25 is 27.5; resulting in the range 22.5-27.5). As an example, a composition comprising “approximately 25 μg” of component A means the composition comprises “22.5-27.5 μg” of component A. As a further example, a composition comprising “about 25-30 μg” of component A means the composition comprises “22.5-33 μg” of component A (10% below 25 is 22.5 and 10% above 30 is 33). As a further example, a composition comprising “approximately 25-30 μg” of component A means the composition comprises “22.5-33 μg” of component A.

“Adjuvant” means an agent that, or composition comprising an agent, that modulates an immune response in a non-specific manner and accelerates, prolongs, and/or enhances the immune response to an antigen. Such an agent may be an “immunostimulant”. An “adjuvant” herein may be a composition that comprises one or more immunostimulants (in particular, an immunostimulating effective amount of one or more immunostimulants (e.g., a saponin)). A “pharmaceutical-grade adjuvant” means an adjuvant suitable for pharmaceutical use (e.g., an adjuvant comprising one or more purified immunostimulant, in particular comprising an immunologically effective amount of a purified immunostimulant). Therefore and for clarity, an adjuvant administered with an antigen produces an accelerated, prolonged, and/or enhanced immune response than the antigen alone does.

The term “and/or” as used in a phrase such as “A and/or B” is intended to include “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone). Similarly, the word “or” is intended to include each of the listed elements individually as well as any combination of the elements (i.e., “or” herein encompasses “and”), unless the context clearly indicates otherwise.

“Antigen” means a molecule, structure, compound, or substance (e.g., a polynucleotides (DNA, RNA), polypeptides, protein complexes) that can stimulate an immune response by producing antigen-specific antibodies and/or an antigen-specific T cell response in a subject (e.g., a human subject). Antigens may be live, inactivated, purified, and/or recombinant. For clarity, an adjuvant is not an antigen at least because an adjuvant cannot (alone) induce antigen-specific immune response. As used herein, an antigen is immunogenic. The term “antigen” includes all related antigenic epitopes. The term “epitope” means that portion of an antigen that determines its immunological specificity and refers to a site on an antigen to which B and/or T cells respond. “Predominant antigenic epitopes” are those epitopes to which a functionally significant host immune response (e.g., an antibody response or a T-cell response) is made. Thus, the predominant antigenic epitopes are those antigenic moieties that, when recognized by the host immune system, result in a protective immune response. The term “T-cell epitope” refers to an epitope that, when bound to an appropriate MHC molecule, is specifically bound by a T cell (via a T cell receptor). A “B-cell epitope” is an epitope that is specifically bound by an antibody (or B cell receptor molecule).

Articles—As used herein, the singular form of articles “a,” “an” and “the” include plural references unless the content clearly dictates otherwise.

Comprising—The term “comprising” encompasses “including” as well as “consisting” e.g. a composition “comprising” X may consist exclusively of X or may include something additional e.g. X+Y. Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus a composition “comprising” X may consist exclusively of X or may include something additional e.g. X+Y.

An “effective amount” means an amount sufficient to cause the referenced outcome. An “effective amount” can be determined empirically and in a routine manner using known techniques in relation to the stated purpose. An “immunologically effective amount”, with respect to an antigen or immunogenic composition, is a quantity sufficient to elicit a measurable immune response in a subject (e.g., 1-100 μg of antigen). With respect to an adjuvant, an “adjuvanting effective amount” or “immunostimulating effective amount” (in the case of an adjuvant that is an immunostimulant) is a quantity sufficient to modulate an immune response (e.g., 1-100 μg of adjuvant). To obtain a protective immune response against a pathogen, it can require multiple administrations of an immunogenic composition. So in the context of, for example, a protective immune response, an “immunologically effective amount” encompasses a fractional dose that contributes in combination with previous or subsequent administrations to attaining a protective immune response.

Empty LNPs—As used in this application, “empty LNPs” are LNPs that do not contain RNA or mRNA or SAM. These LNPs will typically be filled with a liquid material, such as described in the literature for encapsulating mRNA inside of LNPs or for preparing empty LNPs.

Endpoints—Unless specifically stated otherwise, providing a numeric range (e.g., “25-30”) is inclusive of endpoints (i.e., includes the values 25 and 30). An endpoint of a range may be excluded by reciting “exclusive of lower endpoint” or “exclusive of upper endpoint”. Both endpoints may be excluded by reciting “exclusive of endpoints”.

“Human dose” means a dose which is in a volume suitable for human use (“human dose volume”) such as 0.25-1.5 ml. For example, a composition formulated in a volume of about 0.5 ml; specifically a volume of 0.45-0.55 ml; or more specifically a volume of 0.5 ml.

Lipid Nanoparticles (LNPs) LNPs (also sometimes referred to as liposomes) are nanoparticles formed from biodegradable and nontoxic negatively charged lipids (such as phospholipids) that encapsulate the mRNA molecules (such as SAM molecules) used in the present invention. Liposomes and LNPs are often considered to be different from each other. For example, the term “liposomes” is often used to refer to particles that have a lipid bilayer surrounding a core, which is usually an aqueous core. In contrast, some LNPs are micellar-like structures (not lipid bilayers) encapsulating a drug molecules, such as RNA, in an aqueous core. But, unless otherwise expressly indicated or unless clearly understood from the context, when the term “LNPs” is used, it also encompasses “liposomes”.

Nanoparticles—Nanoparticles are small particles (generally on the order of 1-1,000 nanometers) that are used as part of a delivery system for SAM molecules. Nanoparticles include, without limitation, LNPs, CNEs and polymer-based nanoparticles. Nanoparticles include at least the nanoparticles described by Zhao et al, Nanoparticle vaccines, Vaccine, 32 (2014) 327-337.

Nucleic acid, polynucleotide, and oligonucleotide—“Nucleic acid,” “polynucleotide,” and “oligonucleotide” are sometimes used interchangeably and can have similar or overlapping meanings. They are inherently composed of a sequence of nucleotides, each nucleotide comprising a phosphate and a nucleoside, a nucleoside comprising a pentose sugar (e.g. deoxyribose and ribose) and a nucleobase (e.g. a purine comprising adenine or guanine and a pyrimidine comprising cytosine, uracil, N1-methyluracil, and thymine). In some embodiments, the nucleoside (i.e. sugar and nucleobase) can be standard nucleosides (i.e. adenosine, deoxyadenosine, guanosine, deoxyguanosine, thymidine (a.k.a. deoxythymidine), uridine, deoxyuridine, cytidine, or deoxycytidine, or methylates thereof (i.e. 5′-methyluridine) or they may be modified nucleosides (i.e. pseudouridine (a.k.a. 5-(β-D-Ribofuranosyl)pyrimidine-2,4(1H,3H)-dione or 5-[(2S,3R,4S,5R)-3,4-Dihydroxy-5-(hydroxymethyl)oxolan-2-yl]pyrimidine-2,4(1H,3H)-dione, CAS No. 1445-07-4, PubChem CID 15047), N1-methyluridine, N1-methylpseudouridine (a.k.a. 5-[(2S,3R,4S,5R)-3,4-Dihydroxy-5-(hydroxymethyl)oxolan-2-yl]-1-methylpyrimidine-2,4-dione, CAS No. 13860-38-3, PubChem CID 99543), or deoxyribose-containing or ribose-containing forms thereof). In some embodiments, the modified nucleotides comprise hypoxanthine, inosine, 8-oxo-adenine, 7-substituted derivatives thereof, dihydrouracil, pseudouridine, N1-methylpseudouridine, 2-thiouracil, 4-thiouracil, 5-aminouracil, 5-(C1-C6)-alkyluracil, 5-methyluracil, 5-(C2-C6)-alkenyluracil, 5-(C2-C6)-alkynyluracil, 5-(hydroxymethyl)uracil, 5-chlorouracil, 5-fluorouracil, 5-bromouracil, 5-hydroxy cytosine, 5-(C1-C6)-alkylcytosine, 5-methylcytosine, 5-(C2-C6)-alkenylcytosine, 5-(C2-C6)alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, or hydrogen (abasic residue). A “nucleic acid,” “polynucleotide,” and “oligonucleotide” can be a stand-alone molecule (i.e. an RNA molecule) or they may be “region,” “sequence,” or “segment” therein, and in this regard, the use of “region,” “sequence,” or “segment” is used to distinguish between such and a stand-alone molecule.

Process and Method Steps—Unless specifically stated, a process comprising a step of mixing two or more components does not require any specific order of mixing. Thus components can be mixed in any order. Where there are three components then two components can be combined with each other, and then the combination may be combined with the third component, etc.

“Purified” means removed from its natural environment and substantially free of impurities from that natural environment (such as other chromosomal and extra-chromosomal DNA and RNA, organelles, and proteins (including other proteins, lipids, or polysaccharides which are also secreted into culture medium or result from lysis of host cells). For clarity and as used herein, an antigen within a pharmaceutical, immunogenic, vaccine, or adjuvant composition is a purified antigen (whether or not the word “purified” is recited). It is understood in the field that for an antigen, agent, adjuvant, additive, vector, molecule, compound, or composition in general to be suitable for pharmaceutical or vaccine use (i.e., “pharmaceutically acceptable”), it must be purified (i.e., not crude). It would be further understood that “purified” is a relative term and that absolute (100%) purity is not required for, e.g., pharmaceutical or vaccine use. A molecule may be at a purity of at least 85%, 86%, 87%, 88%, 89%, 90%, 91%, 92%, 93%, 94% or 95% of a composition's total proteinaceous mass (determined by, e.g., gel electrophoresis). Methods of purification are known and include, e.g., various types of chromatography such as High Performance Liquid Chromatography (HPLC), hydrophobic interaction, ion exchange, affinity, chelating, and size exclusion; electrophoresis; density gradient centrifugation; or solvent extraction. “Isolated” means removed from its natural environment and not linked to a recombinant molecule or structure (e.g., not bound to a recombinant antibody or antibody fragment) including not linked to a laboratory tool (e.g., not linked to a chromatography tool such as not bound to an affinity chromatography column). Hence, an “isolated betacoronavirus antigen”, such as an “isolated modified betacoronavirus Spike protein or Spike protein fragment”, is not on the surface of a betacoronavirus-infected cell or within an infectious betacoronavirus virion or bound to a recombinant antibody or recombinant antibody fragment (which occurs in an ELISA assay, for example). It would be understood that an antigen being bound to an antibody or antibody fragment (through epitope recognition, for example) is different than an antigen being operably linked to an antibody or antibody fragment (operable linkage in that case would use recombinant techniques and produces a molecule that does not occur in nature).

“Recombinant” when used to describe a biological molecule or biological structure (e.g., protein, nucleic acid, organism, cell, vesicle, sacculi, or membrane) means the biological molecule or biological structure is artificially produced (e.g., by laboratory methods), synthetic, and/or has a different structure and/or function than the molecule or structure from which it was obtained or than its wild type counterpart. For clarity, a recombinant molecule or recombinant structure that is synthetic may nonetheless function comparably to its wild type counterpart. For clarification, a “recombinant nucleic acid” or “recombinant polynucleotide” means a nucleic acid/polynucleotide that, by virtue of its origin or manipulation (e.g., by laboratory methods), (1) is not associated with all or a portion of the polynucleotide with which it is associated in nature; and/or (2) is linked to a polynucleotide other than that to which it is linked in nature. A “recombinant protein/polypeptide” thereby encompasses a protein/polypeptide produced by expression of a recombinant polynucleotide. For clarification, a “purified protein” (e.g., a protein suitable for pharmaceutical use) is encompassed within the term “recombinant protein” because a purified protein is both artificially produced and has a different function than the crude protein (or extract or culture) from which it was obtained. A biological molecule or biological structure of the present invention may be described as “artificially produced”.

“Heterologous” denotes that the two referenced biological molecules or biological structures are not naturally associated with each other (would not contact each other but—for the hand of man) or that the referenced biological molecule/structure is not in its natural environment. For example, when a nucleic acid molecule is operably linked to another polynucleotide that it is not associated with in nature, the nucleic acid molecule may be referred to as “heterologous” (i.e., the nucleic acid molecule is heterologous to at least the polynucleotide). Similarly, when a polypeptide is in contact with or in a complex with another protein that it is not associated with in nature, the polypeptide may be referred to as “heterologous” (i.e., the polypeptide is heterologous to the protein). Further, when a host cell comprises a nucleic acid molecule or polypeptide that it does not naturally comprise, the nucleic acid molecule and polypeptide may be referred to as “heterologous” (i.e., the nucleic acid molecule is heterologous to the host cell and the polypeptide is heterologous to the host cell).

mRNA-LNPs—The term mRNA-LNPs refers to LNPs that contain mRNA therein. In the context of the present invention, the mRNA will encode a desired antigen. The mRNA can be mRNA such as Self-Amplifying mRNA or a non-replicating mRNA.

SAM (Self-Amplifying mRNA or SAM replicon) or SAM Molecules—SAM or SAM Molecules useful in accordance with the present invention contain at least mRNA that encodes an immunogenic or therapeutic polypeptide that elicits a preventative or therapeutic immune response or a therapeutic response against infection with a pathogen. The SAM molecule includes elements that allow the mRNA to self-replicate in vitro in a cell and/or also to replicate in vivo in an animal and/or human. In aspects, the SAM molecule does not encode structural virion proteins.

SAM RNA Vaccine—A SAM RNA vaccine is a product that comprises a SAM Molecule, formulated with a delivery vehicle, that can elicit an immunological response when administered to a patient.

Substantially—The word “substantially” does not exclude “completely” e.g. a composition which is “substantially free” from Y may be completely free from Y. Where necessary, the word “substantially” may be omitted from the definition of the invention.

Units of mass and concentration—As used herein, ng refers to nanograms, ug or μg refers to micrograms, mg refers to milligrams, mL or ml refers to milliliter, and mM refers to millimolar. Similar terms, such as um, are to be construed accordingly.

### Embodiments of the Invention

Various numbered embodiments of the present invention are described below, but the invention is not limited to these embodiments.
1. A method of preparing a low-dose lyophilized RNA immunogenic composition, comprising:
forming a mixed aqueous composition comprising RNA containing lipid nanoparticles (RNA-LNPs), wherein said RNA encodes at least one immunogen, and empty lipid nanoparticles (empty LNPs); and
lyophilizing said mixed aqueous composition.
2. The method of embodiment 1, wherein said RNA is mRNA and said RNA-LNPs are mRNA-containing lipid nanoparticles (mRNA-LNPs).
3. The method of embodiment 1 or embodiment 2, wherein said RNA is self-amplifying mRNA (SAM) and said RNA-LNPs are SAM-containing lipid nanoparticles (SAM-LNPs).
4. The method of embodiment 3, wherein a composition containing said SAM-LNPs and a separate composition containing said empty LNPs are mixed to form said mixed aqueous composition and said mixed aqueous composition is thereafter lyophilized.
5. The method of embodiment 4, wherein said empty LNPs are present in an effective amount up to 25% dry weight of lipids based on the total dry weight of lipids in said empty LNPs and the total dry weight of lipids in said SAM-LNPs.
6. A vaccine composition comprising the immunogenic composition of any of the preceding embodiments.
7. A low-dose lyophilized immunogenic composition, comprising:
RNA containing lipid nanoparticles (RNA-LNPs), wherein said RNA encodes at least one immunogen; and
empty lipid nanoparticles (empty LNPs).
8. The low-dose lyophilized immunogenic composition of embodiment 7, wherein said RNA is mRNA and said RNA-LNPs are mRNA-containing lipid nanoparticles (mRNA-LNPs).
9. The low-dose lyophilized immunogenic composition of embodiment 7 or embodiment 8, wherein said RNA is self-amplifying mRNA (SAM) and said RNA-LNPs are SAM-containing lipid nanoparticles (SAM-LNPs).
10. The low-dose lyophilized immunogenic composition of any of embodiments 7-9, wherein said low-dose immunogenic composition can be reconstituted with a liquid for reconstitution to prepare an injectable vaccine composition that is ready for injection, without further dilution after reconstitution.
11. The low-dose lyophilized immunogenic composition of embodiment 9, having an amount of SAM of less than 60 μg of SAM that is encapsulated in LNPs to form SAM-LNPs, said amount of SAM being based on a single dose of a SAM-LNP low-dose lyophilized vaccine composition.
12. The low-dose lyophilized immunogenic composition of embodiment 10, having an amount of SAM of 1 to 6 μg of SAM that is encapsulated in LNPs to form SAM-LNPs, said amount of SAM being based on a single dose of a SAM-LNP low-dose lyophilized vaccine composition.
13. The low-dose lyophilized immunogenic composition of any of embodiments 7-12, wherein said low-dose lyophilized immunogenic composition comprises, in addition to components that form said LNPs and in addition to said RNA, one or more of the following additional dry components; buffer, sodium chloride and sugar.
14. The lyophilized immunogenic composition of any of embodiments 7-13, wherein said low-dose lyophilized vaccine comprises, in addition to components that form said LNPs and in addition to said RNA, a dry buffer component such that when the lyophilized vaccine composition is mixed with sterile water for injection an injectable aqueous pharmaceutically acceptable aqueous composition is formed.
15. A vaccine composition comprising the lyophilized immunogenic composition of any of embodiments 7-14.
16. An injectable vaccine composition made by reconstituting the vaccine composition of embodiment 15.
17. A method for making an injectable vaccine composition, comprising:
mixing a lyophilized vaccine composition comprising said immunogenic composition of any of embodiments 7-15 with an aqueous liquid for reconstitution to form a reconstituted composition, and optionally further diluting said reconstituted composition, to form an aqueous injectable vaccine composition.
18. A method for administering a low-dose vaccine to a subject, comprising:
reconstituting said lyophilized vaccine composition of any of embodiments 7-15 with a liquid for reconstitution to make a reconstituted composition; and
administering a low-dose vaccine comprising said reconstituted composition to said subject.
19. A method for administering a low-dose vaccine to a subject, comprising:
reconstituting said lyophilized vaccine composition of embodiment 15, with a liquid for reconstitution to make a reconstituted composition; and
administering a low-dose vaccine comprising said reconstituted composition to said subject without any further dilution after reconstitution.
20. A low-dose vaccine kit, comprising:
the lyophilized low-dose vaccine composition of embodiment 15 in a container; and
a sterile needle for injecting a vaccine composition and/or a second container containing a sterile aqueous solution and/or an adjuvant.
21. The low-dose vaccine kit of embodiment 20, wherein said container comprises enough of said lyophilized low-dose vaccine composition to make multiple low-doses of said low-dose vaccine.
22. A low-dose lyophilized RNA vaccine made by the method of embodiment 1, 2, 3, 4 or 5.
23. The vaccine composition of embodiment 15, for use eliciting an immune response in a patient to which said vaccine composition is administered.
24. The injectable vaccine composition of embodiment 16, for use eliciting an immune response in a patient to which said vaccine composition is administered.
25. The use of the vaccine composition of embodiment 15 or embodiment 16 for the manufacture of a medicament for use in inducing an acceptable immune response in a subject, wherein the medicament is prepared to be administered by injection.

### EXAMPLES

The following procedures may be used to prepare, lyophilize, and reconstitute mRNA-LNPs as well as assess physical properties in the following Examples.

### Example 1—Preparation of LNPs

Formation of LNPs that Encapsulate RNA

RNA-LNPs may be formulated according to the following procedures. Techniques for generating RNA replicons are also summarized as follows. In some aspects, RNA may be in the form of RNA replicons. In other aspects, RNA may be in the form of non-replicating RNA.

Replicons may be based on a hybrid alphavirus genome with non-structural proteins from Venezuelan equine encephalitis virus (VEEV), a packaging signal from Sindbis virus, and a 3′ UTR from Sindbis virus or a VEEV mutant. The replicon is about 10 kb long and has a poly-A tail.

Plasmid DNA encoding alphavirus replicons (named: pT7-mVEEV-FL.RSVF or A317; pT7-mVEEV-SEAP or A306; pSP6-VCR-GFP or A50) served as a template for synthesis of RNA in vitro. The replicons contain the alphavirus genetic elements required for RNA replication but lack those encoding gene products necessary for particle assembly; the structural proteins are instead replaced by a protein of interest (either a reporter, such as SEAP or GFP, or an immunogen, such as full-length RSV F protein) and so the replicons are incapable of inducing the generation of infectious particles. A bacteriophage (T7 or SP6) promoter upstream of the alphavirus cDNA facilitates the synthesis of the replicon RNA in vitro and a hepatitis delta virus (HDV) ribozyme immediately downstream of the poly(A)-tail generates the correct 3′-end through its self-cleaving activity.

Following linearization of the plasmid DNA downstream of the HDV ribozyme with a suitable restriction endonuclease, run-off transcripts were synthesized in vitro using T7 or SP6 bacteriophage derived DNA-dependent RNA polymerase. Transcriptions were performed for 2 hours at 37° C. in the presence of 7.5 mM (T7 RNA polymerase) or 5 mM (SP6 RNA polymerase) of each of the nucleoside triphosphates (ATP, CTP, GTP and UTP) following the instructions provided by the manufacturer (Ambion). Following transcription the template DNA was digested with TURBO DNase (Ambion). The replicon RNA was precipitated with LiCl and reconstituted in nuclease-free water. Uncapped RNA was capped post-transcriptionally with Vaccinia Capping Enzyme (VCE) using the ScriptCap m7G Capping System (Epicentre Biotechnologies) as outlined in the user manual; replicons capped in this way are given the “v” prefix e.g. vA317 is the A317 replicon capped by VCE. Post-transcriptionally capped RNA was precipitated with LiCl and reconstituted in nuclease-free water. The concentration of the RNA samples was determined by measuring OD₂₆₀ₙₘ. Integrity of the in vitro transcripts was confirmed by denaturing agarose gel electrophoresis.

In the experiments reported in these Examples, the mRNA that is encapsulated is SAM-LNP that encodes a COVID-19 spike protein. SAM molecules that encode a COVID spike protein may be referred to herein as “COVID SAM”.

### Liposomal Encapsulation

In the Examples, the physical structure of the LNPs was not determined to confirm whether or not they contain a lipid bilayer (like traditional LNPs) or whether or not they are more like “LNPs” (not containing a lipid bilayer). In this regard, see the definition of LNPs above. It is quite possible, that the empty LNPs may tend towards having a lipid bilayer structure, although all of the LNPs may not have this structure. Similarly, it is possible that RNA-LNPs, mRNA-LNPs or SAM-LNPs may tend more towards not having a lipid bilayer structure, but this has not confirmed. Therefore, the terms “LNPs” and “liposomes” used in the following experimental procedures should be interpreted with this in mind, with the understanding that the structure of these LNPs has not been fully confirmed and there may be some heterogeneity within the populations of RNA-LNPs, mRNA-LNPs or SAM-LNPs and empty LNPs in terms of whether or not they contain a lipid bilayer.

RNA was encapsulated in liposomes made by the method of El Ouahabi et al. (1996) FEBS Letts 380:108-12and Maurer et al. (2001) Biophysical Journal, 80: 2310-2326. The liposomes were made of 10% DSPC (zwitterionic), 40% DlinDMA (cationic), 48% cholesterol and 2% PEG-conjugated DMG (2 kDa PEG). These proportions refer to the % moles in the total liposome.

DlinDMA (1,2-dilinoleyloxy-N,N-dimethyl-3-aminopropane) was synthesized using the procedure of EP 2 591 103. DSPC (1,2-Diastearoyl-sn-glycero-3-phosphocholine) was purchased from Genzyme. Cholesterol was obtained from Sigma-Aldrich. PEG-conjugated DMG (1,2-dimyristoyl-sn-glycero-3-phosphoethanolamine-N-[methoxy(polyethylene glycol), ammonium salt), DOTAP (1,2-dioleoyl-3-trimethylammonium-propane, chloride salt) and DC-chol (3β-[N—(N′,N′-dimethylaminoethane)-carbamoyl]cholesterol hydrochloride) were from Avanti Polar Lipids.

Briefly, lipids were dissolved in ethanol (2 ml), a RNA replicon was dissolved in buffer (2 ml, 100 mM sodium citrate, pH 6) and these were mixed with 2 ml of buffer followed by 1 hour of equilibration. The mixture was diluted with 6 ml buffer then filtered. The resulting product contained liposomes, with ˜95% encapsulation efficiency.

For example, in one particular method, fresh lipid stock solutions were prepared in ethanol. 37 mg of DlinDMA, 11.8 mg of DSPC, 27.8 mg of cholesterol and 8.07 mg of PEG-DMG were weighed and dissolved in 7.55 mL of ethanol. The freshly prepared lipid stock solution was gently rocked at 37° C. for about 15 min to form a homogenous mixture. Then, 755 μL of the stock was added to 1.245 mL ethanol to make a working lipid stock solution of 2 mL. This amount of lipids was used to form liposomes with 250 μg RNA. A 2 mL working solution of RNA was also prepared from a stock solution of ˜1 μg/μL in 100 mM citrate buffer (pH 6). Three 20 mL glass vials (with stir bars) were rinsed with RNase Away solution (Molecular BioProducts) and washed with plenty of MilliQ water before use to decontaminate the vials of RNases. One of the vials was used for the RNA working solution and the others for collecting the lipid and RNA mixes (as described later). The working lipid and RNA solutions were heated at 37° C. for 10 min before being loaded into 3 cc luer-lok syringes. 2 mL citrate buffer (pH 6) was loaded in another 3 cc syringe. Syringes containing RNA and the lipids were connected to a T mixer (PEEK™ 500 μm ID junction, Idex Health Science) using FEP tubing (fluorinated ethylene-propylene; all FEP tubing used had a 2 mm internal diameter and a 3 mm outer diameter; obtained from Idex Health Science). The outlet from the T mixer was also FEP tubing. The third syringe containing the citrate buffer was connected to a separate piece of tubing. All syringes were then driven at a flow rate of 7 mL/min using a syringe pump. The tube outlets were positioned to collect the mixtures in a 20 mL glass vial (while stirring). The stir bar was taken out and the ethanol/aqueous solution was allowed to equilibrate to room temperature for 1 h. 4 ml of the mixture was loaded into a 5 cc syringe, which was connected to a piece of FEP tubing and in another 5 cc syringe connected to an equal length of FEP tubing, an equal amount of 100 mM citrate buffer (pH 6) was loaded. The two syringes were driven at 7 mL/min flow rate using the syringe pump and the final mixture collected in a 20 mL glass vial (while stirring). Next, the mixture collected from the second mixing step (liposomes) were passed through a Mustang Q membrane (an anion-exchange support that binds and removes anionic molecules, obtained from Pall Corporation). Before using this membrane for the liposomes, 4 mL of 1 M NaOH, 4 mL of 1 M NaCl and 10 mL of 100 mM citrate buffer (pH 6) were successively passed through it. Liposomes were warmed for 10 min at 37° C. before passing through the membrane. Next, liposomes were concentrated to 2 mL and dialyzed against 10-15 volumes of 1×PBS using by tangential flow filtration before recovering the final product. The TFF system and hollow fiber filtration membranes were purchased from Spectrum Labs (Rancho Dominguez) and were used according to the manufacturer's guidelines. Polysulfone hollow fiber filtration membranes with a 100 kD pore size cutoff and 8 cm² surface area were used. For in vitro and in vivo experiments formulations were diluted to the required RNA concentration with 1×PBS.

Alternatively, liposomes suitable for use herein may be made according to the Examples of WO2018220553.

The liposomes may be concentrated and dialyzed against any suitable buffer (e.g., Histidine, Tris, Citrate, HEPES, etc.).

### Formulation

Stock solutions of various components, as shown in Table 2, were generated including: sucrose (20% w/v), secondary sugars (e.g., glucose 20% w/v, trehalose 20% w/v, maltose 20% w/v, and melezitose 20% w/v), amino acids (e.g., arginine 10% w/v, methionine 4% w/v, histidine 4% w/v, lysine 10% w/v, and alanine 10% w/v), and plasticizers (e.g., glycerol 20% w/v and sorbitol at 20% w/v).

TABLE 2

| | Stock | | | Final |
|---|---|---|---|---|
| | Conc. (% | | Amount | volume |
| Excipient | w/v) | Weight (g) | weighed (g) | (mL) |
| Sucrose | 20 | 10 | 10.02 | 50 |
| Glucose | 20 | 5 | 5.04 | 25 |
| Trehalose | 20 | 5 | 5.03 | 25 |
| Maltose | 20 | 5 | 5.00 | 25 |
| Melezitose | 20 | 1 | 1.00 | 5 |
| Histidine | 4 | 0.4 | 0.40 | 10 |
| Methionine | 4 | 0.4 | 0.41 | 10 |
| Glycerol | 20 | 2 | 2.01 | 10 |
| Sorbitol | 20 | 2 | 2.00 | 10 |

All excipients were filtered using a PES membrane filter. Sugars were filtered using a 50 mL, 0.22 um Millipore filter. Excipients 10 mL or less were filtered using a 0.45 um syringe filter.

### Buffer Preparation

1) Amounts were weighed on weigh boats as indicated below and transferred to a 1 L volumetric flask.

TABLE 3

| Buffer Components | Quantities needed (g) | Amount weighed (g) |
|---|---|---|
| Vol. of buffer needed | 1 | 1 |
| Tris(hydroxymethyl)aminomethane = | 1.06 | 1.06012 |
| 1.06 *x (g) = | | |
| Tris(hydroxymethyl)aminomethane | 1.78 | 1.7805 |
| hydrochloride = 1.78 * x (g) = | | |
| Sodium Chloride = 0.29 * x (g) = | 0.29 | 0.294 |

2) Sterile water was added to the volumetric flask sufficient to the 1 L mark, and the flask was stirred for ˜10 mins. The pH was measured to ensure 8.0±0.1. The volume was adjusted to 1 L and filtered using a Millipore 0.22 um PES filter (e.g., 0.45 um PES, L/N 1274753, ThermoFisher).

A protocol for preparation of stock solution is provided as follows.

A stock SAM-LNP buffer (20 mM Tris with 5 mM NaCl —NO SUCROSE) was made. Stock solutions as shown in Table 2 were obtained in the corresponding LNP buffer. Vial labels and Eppendorf tube labels were made. The gB SAM-LNP stock was thawed. Buffer exchange was performed using a PD10 column to remove sucrose according to a spin protocol (e.g., Spin Protocol of PD-10 Desalting Columns of Instructions 52-1308-00 BB; GE Healthcare). Formulations were prepared according to calculated volumes. Two vials were filled for each formulation and placed on the tray.

The stock solution from Table 2 is incorporated into Table 3, to keep the final volume of the formulation consistent. In this case, the concentration of SAM-LNPs was kept unchanged after addition of all excipients.

Different compositions were evaluated after lyophilization and CQAs (e.g., size, PLI, % encapsulation) were measured and compared to a control formulation (e.g., a lyophilized control formulation and a pre-lyophilized control formulation) according to the techniques provided herein.

### Lyophilization

The lyophilization process was designed to maximize percent encapsulation of mRNA in LNPs and to preserve the percent encapsulation such that it is comparable to a liquid pre-lyophilization control. The lyophilization device is LyoStar3 by SP Scientific. Lyophilization steps included loading, thermal equilibrium, freezing, primary drying, and secondary drying.

The lyophilization process was carried out according to the controlled parameters of cycle time, step order, temperature, pressure as well as ramp rate. These parameters controlled the environment of the lyophilization chamber for each step.

A lyophilization process is shown according to the embodiments provided herein. A sample loading step is performed in which the lyophilization formulation, at room temperature, is loaded into the lyophilization chamber. A first thermal equilibrium cycle is performed with a ramp rate of 1° C. per minute to reach a temperature of 5° C. for a duration of 0.5 hours. Once completing the first step of thermal equilibrium, a second step of thermal equilibrium was performed. The second step of thermal equilibrium was performed with a ramp rate of 1° C. per minute to reach a temperature of minus 5° C. for a duration of 0.5 hours.

A freezing step, for a duration of 1 hour, was performed with a ramp rate of 1° C. per minute to reach a temperature of −40° C. Once completing the freezing step, a primary drying step was performed for a duration of 27 hours, with a ramp rate of 0.5° C. per minute to reach a temperature of minus 29° C. A secondary drying step followed the primary drying step. The secondary drying step was performed for a duration of 12 hours, with a ramp rate of 0.1° C. per minute to reach a temperature of 15° C.

During the lyophilization process, samples were withdrawn using a sample thief, starting from the end of primary drying through the end of lyophilization. For example, in some aspects, samples were removed every two hours and percent encapsulation tested.

Surprisingly, it was found that different secondary drying temperatures and durations impacted the percentage entrapment post lyophilization. In some cases, selected combinations of secondary drying temperatures and time durations resulted in an improvement of percent encapsulation, as the percent encapsulation trended towards its initial value.

### Evaluation CQAs for Lyophilized Product

For both the process and formulation development of components and buffers, pre and post lyophilization samples were analyzed for size (e.g., using Dynamic Light Scattering (DLS) to determine hydrodynamic diameter and Polydispersity Index (PDI)), and percent encapsulation (e.g., using RiboGreen Fluorescence or DLS (Wyatt & Malvern) assay. RNA integrity was assessed by agarose gel electrophoresis. For select samples, lipid content was determined (by Reverse Phase HPLC) and moisture content (Karl Fisher).

### Dynamic Light Scattering

Liposome size can be determined in various ways (e.g., using the techniques of dynamic light scattering and/or single-particle optical sensing, using an apparatus such as the Accusizer™ and Nicomp™ series of instruments available from Particle Sizing Systems (Santa Barbara, USA), the Zetasizer™ instruments from Malvern Instruments (UK), or the Particle Size Distribution Analyzer instruments from Horiba (Kyoto, Japan)). See, Light Scattering from Polymer Solutions and Nanoparticle Dispersions Schartl, 2007. Dynamic light scattering (DLS) is the preferred method by which liposome size is determined. The preferred method for defining the average liposome diameter is a Z-average i.e. the intensity-weighted mean hydrodynamic size of the ensemble collection of liposomes measured by DLS. The Z-average is derived from cumulants analysis of the measured correlation curve, wherein a single particle size (droplet diameter) is assumed and a single exponential fit is applied to the autocorrelation function. Thus, references herein to average particle size should be taken as an intensity-weighted average, and ideally the Z-average. PDI values are easily provided by the same instrumentation which measures average diameter.

### Determination of % Encapsulation

The percentage of encapsulated RNA and RNA concentration, in SAM-LNP compositions, were determined by Quant-iT RiboGreen RNA reagent kit (Invitrogen), following manufacturer's instructions. The ribosomal RNA standard provided in the kit was used to generate a standard curve. Liposomes were diluted 10× or 100× in 1× TE buffer (from kit) before addition of the dye. Separately, liposomes were diluted 10× or 100× in 1× TE buffer containing 0.5% Triton X before addition of the dye (to disrupt the liposomes and thus to assay total RNA). Thereafter an equal amount of dye was added to each solution and then ˜180 μL of each solution after dye addition was loaded in duplicate into a 96 well tissue culture plate. The fluorescence (Ex 485 nm, Em 528 nm) was read on a microplate reader. All liposome formulations may be dosed in vivo based on the encapsulated amount of RNALiposome doses are stated based on the calculated or estimated encapsulated amount of RNA, mRNA or SAM.

### Determining Percent of Empty LNPs

In the Examples below, the number of empty LNPs was not directly assayed. Rather, the “amount” of empty LNPs was reported based on the amount (mass) of lipid in an LNP formulation. For SAM-LNPs, the amount of lipid in a sample can be determined based on the preparation method. Similarly, the amount of lipid in an empty LNP sample can be determined based on the preparation. In the Examples, the amount of SAM-LNPs and empty LNPs was reported based on the amount of lipid in each preparation, prior to mixing. The percent of empty LNPs in a sample (such as a mixed sample) was calculated using these two values.

It is believed that an accurate estimation of the percent of the “number” of empty LNPs (the number of empty LNPs divided by the total number of empty LNPs and the total number of SAM-LNPs) will be about the same as the corresponding “amount” based on the following amount of lipid will be about the same where the following assumptions are true: (1) the size of a typical LNP in an empty LNP composition will be approximately the same as the average size of a SAM-LNP; (2) the amount (mass) of total lipids in a typical LNP in an empty LNP composition will be approximately the same as the amount of total lipids in a typical SAM-LNP; and (3) substantially all of the LNPs in the SAM-LNP composition will have at least one SAM molecule contained therein. Thus, by knowing the total dry weight of lipids in each composition, the percent of empty LNPs in a mixed composition can be estimated, as mentioned above. Thus, the “amount” or “percentage” of empty LNP based on weight of lipids will be about the same as the “number” of empty LNPs.

### Buffer Variation

SAM-LNPs were formulated in PBS and concentrated as described above. For evaluation of compositions in various buffers, such as Tris, histidine, citrate, and HEPES buffer, LNPs in PBS were subjected to buffer exchange and/or desalting (e.g., such as by size exclusion chromatography) according to techniques known in the art.

Traditionally, it has been thought that acidic buffers such as citrate buffer or histidine buffer would not be suitable for lyophilization of LNP-mRNA due to decreased LNP stability. For example, for histidine buffer, which has a pKa of about 6.4, it was thought that the LNP would be less stable in acidic conditions.

### In Vitro Potency

It is desirable for the vaccine composition to have the same or very similar In vitro potency before lyophilization and after reconstitution (and after dilution if dilution is performed). Any in vitro assay can be used to measure in vitro potency. The following exemplary assay is described as one option.

### Lyophilizing Low-Dose SAM-LNP—Formulation

To lyophilize low-dose SAM-LNP, a tube of frozen SAM-LNP was thawed and diluted serially to doses 120, 60, 30, 10, 5, 2 and 1 μg/mL SAM-LNP. Another tube of frozen empty LNPs was thawed, and these empty LNPs were added to the diluted SAM-LNPs at varying concentrations. Empty LNPs are formulated using the same process as SAM-LNPs such that the aqueous phase contains the citrate buffer at pH 6 without the SAM. These LNPs are then buffer exchanged into 20 mM Tris, 5 mM NaCl and 7.5% Sucrose buffer to remove ethanol and allow LNPs to mature. 0.7 mL of a mixture containing both the SAM-LNPs and the empty LNPs was then added to each vial and subjected to freeze-drying using the optimized lyophilization process described above. The following Table 4 represents a typical serial dilution of the SAM-LNPs and the Empty LNP, including control formulations.

TABLE 4

| Formulation | SAM-LNP conc. | Empty LNP con. |
|---|---|---|
| number | (μg/mL) | (μg/mL) |
| Control -1 | 60 | N/A |
| Control - 2 | N/A | 60 |
| 3 | 120 | 30 |
| 4 | 60 | 30 |
| 5 | 30 | 30 |
| 6 | 10 | 30 |
| 7 | 5 | 30 |
| 8 | 2 | 30 |
| 9 | 1 | 30 |

### Example 2—Low-Dose Sam-LNP

The following Table 5 describes samples of COVID SAM were prepared in a buffer of 7.5% sucrose, 20 mM Tris and 5 mM NaCl.

TABLE 5

| | Theoretical Conc. | | |
|---|---|---|---|
| Sample | (ug/mL) | % E | Size (DLS) |
| 10 ug/mL | 10 | 53.52 | 113.67 |
| 20 ug/mL | 20 | 59.91 | 113.57 |
| 30 ug/mL | 30 | 61.03 | 114.83 |
| 60 ug/mL | 60 | 59.58 | 116.30 |
| 80 ug/mL | 80 | 63.40 | 116.97 |
| 120 ug/mL | 120 | 62.44 | 118.13 |
| 180 ug/mL | 180 | 63.50 | 119.57 |
| Control 1 | 190 | 64.90 | 119.77 |
| liq. control | 190 | 88.50 | 112 |

### Example 3—Constant and Increasing Empty LNP Concentration

Table 6 reports various properties of Formulations 1-9 which have varying amounts of SAM-LNP and compares these values against theoretical concentration of LNPs after adding empty LNPs. The percent encapsulation of the SAM in the LNPs (% E) and concentration of SAM/mRNA were determined by Ribogreen Fluorescence and the average size of the LNPs and the average PDL of the LNPs were determined by DLS. The theoretical concentration of all LNPs (after adding empty LNPs) is also reported.

TABLE 6

| | | | Theoretical | | | DLS | |
|---|---|---|---|---|---|---|---|
| | SAM- | Empty | Conc. of | | | Avg | Avg |
| | LNP | LNP | LNPs after | Ribogreen | | Size | PdI |
| Form. | con. | conc. | adding empty | | Conc. | by DLS | by DLS |
| # | (μg/mL) | (μg/mL) | (μg/mL) | % E | (ug/mL) | (nm) | (nm) |
| SAM- | 252 | N/A | 252 | 72.9 | 221.4 | 122.1 | 0.116 |
| LNP | | | | | | | |
| control | | | | | | | |
| 1 | | | | | | | |
| Empty | N/A | 186 | 186 | −88.9 | −88.94 | 114.4 | 0.111 |
| LNP | | | | | | | |
| control | | | | | | | |
| 2 | | | | | | | |
| 3 | 120 | 60 | 180 | 73.0 | 100.85 | 116.9 | 0.109 |
| 4 | 60 | 60 | 120 | 74.7 | 51.66 | 113.5 | 0.117 |
| 5 | 30 | 60 | 90 | 75.8 | 25.63 | 112.3 | 0.106 |
| 6 | 20 | 60 | 80 | 75.5 | 16.32 | 109.7 | 0.112 |
| 7 | 10 | 60 | 70 | 78.1 | 9.82 | 109.1 | 0.108 |
| 8 | 5 | 60 | 65 | 76.7 | 4.51 | 108.0 | 0.128 |
| 9 | 2 | 60 | 62 | 74.6 | 2.07 | 109.0 | 0.112 |

Table 7 reports the same properties as Table 6 for Formulations 10-16 and a Liquid Control which is a frozen liquid control at high concentration. The high concentration of a liquid control is usually between 120 and 220 μg/ml of RNA in SAM-LNPs.

TABLE 7

| | | | Theoretical | | | DLS | |
|---|---|---|---|---|---|---|---|
| | SAM- | Empty | Conc. of | | | Avg | Avg |
| | LNP | LNP | LNPs after | Ribogreen | | Size | PdI |
| Form. | conc. | con. | adding empty | | Conc. | by DLS | by DLS |
| # | (μg/mL) | (μg/mL) | (μg/mL) | % E | (ug/mL) | (nm) | (nm) |
| 10 | 142 | 105 | 246 | 74.9 | 107.8 | 118.6 | 0.111 |
| 11 | 71 | 134 | 205 | 74.6 | 73.12 | 117.6 | 0.132 |
| 12 | 35 | 160 | 195 | 76.9 | 29.20 | 116.7 | 0.134 |
| 13 | 18 | 173 | 191 | 76.8 | 15.18 | 115.6 | 0.123 |
| 14 | 9 | 179 | 188 | 76.4 | 7.56 | 115.0 | 0.138 |
| 15 | 4 | 183 | 187 | 74.7 | 4.13 | 116.3 | 0.132 |
| 16 | 2 | 184 | 187 | 74.6 | 2.13 | 114.7 | 0.122 |
| Liq. | 252 | N/A | 252 | 87.3 | 224.2 | 109.8 | 0.075 |
| control | | | | | | | |

Tables 6 and 7 show that addition of empty LNPs to low-dose SAM-LNPs may protect loss in entrapment. For example, 60 μg/ml of empty LNPs seem sufficient to maintain the % encapsulation for low-dose SAM-LNPs comparable to a lyophilized control (lyo control, which is Formulation #1 in Table 6).

### Tables 8 and 9

Tables 8 and 9 confirm that addition of empty LNPs to low-dose SAM-LNPs protects loss in entrapment. 30 μg/ml of empty LNPs are comparable to 60 μg/ml suggesting that a small number of empty particles are needed to stabilize low-dose SAM-LNP during the freeze drying process.

TABLE 8

| | | | Theoretical | | | | |
|---|---|---|---|---|---|---|---|
| | | | Conc. of | | | | |
| | SAM- | Empty | LNPs after | | | | |
| | LNP | LNP | adding | Ribogreen | | DLS | |
| Form. | conc. | con. | empty | | Conc. | Size | PdI |
| # | (μg/mL) | (μg/mL) | (μg/mL) | % E | (ug/mL) | (nm) | (nm) |
| 1 | 252 | N/A | 252 | 69.57 | 232.02 | 124.17 | 0.104 |
| 2 | N/A | 186 | 186 | 406.43 | 0.79 | 115.83 | 0.141 |
| 3 | 120 | 60 | 180 | 70.88 | 106.43 | 117.97 | 0.115 |
| 4 | 60 | 60 | 120 | 67.70 | 49.71 | 115.17 | 0.114 |
| 5 | 30 | 60 | 90 | 69.06 | 24.72 | 113.57 | 0.11 |
| 6 | 20 | 60 | 80 | 72.46 | 17.67 | 113.37 | 0.113 |
| 7 | 10 | 60 | 70 | 74.16 | 9.82 | 112.47 | 0.134 |
| 8 | 5 | 60 | 65 | 74.32 | 4.08 | 111.57 | 0.116 |
| 9 | 2 | 60 | 62 | 73.53 | 1.75 | 111.3 | 0.136 |
| 10 | 1 | 60 | 61 | 74.73 | 0.71 | 110.77 | 0.128 |
| Liq. | 252 | N/A | 252 | 88.72 | 230.34 | 110.1 | 0.068 |
| control | | | | | | | |

TABLE 9

| | | | Theoretical | | | | |
|---|---|---|---|---|---|---|---|
| | | | Conc. of | | | | |
| | SAM- | Empty | LNPs after | | | | |
| | LNP | LNP | adding | Ribogreen | | DLS | |
| Form. | conc. | con. | empty | | Conc. | Size | PdI |
| # | (μg/mL) | (μg/mL) | (μg/mL) | % E | (ug/mL) | (nm) | (nm) |
| 11 | 120 | 30 | 150 | 74.58 | 117.75 | 116.5 | 0.12 |
| 12 | 60 | 30 | 90 | 77.53 | 58.58 | 114.97 | 0.118 |
| 13 | 30 | 30 | 60 | 75.16 | 27.11 | 113.17 | 0.104 |
| 14 | 20 | 30 | 50 | 84.67 | 20.04 | 111.9 | 0.121 |
| 15 | 10 | 30 | 40 | 81.66 | 9.83 | 112.17 | 0.119 |
| 16 | 5 | 30 | 35 | 82.53 | 4.43 | 109.97 | 0.122 |
| 17 | 2 | 30 | 32 | 81.80 | 2.07 | 109.37 | 0.128 |

Table 10—Constant Empty LNP at 60 μg/mL, 30 μg/ml abd 15 μg/ml

Table 10 provides more data (similar to Tables 8 and 9) that confirm that addition of empty LNPs to low-dose SAM-LNPs protects loss in entrapment. 30 μg/ml of empty LNPs are comparable to 60 μg/ml suggesting that a small number of empty particles are needed to stabilize low-dose SAM-LNP during the freeze drying process.

TABLE 10

| | | | Theoretical | | | | |
|---|---|---|---|---|---|---|---|
| | SAM- | Empty | Conc. of | | | DLS | |
| | LNP | LNP | LNPs after | Ribogreen | | Avg Size | Avg PdI |
| Form. | conc. | con. | adding empty | | Conc. | by DLS | by DLS |
| # | (μg/mL) | (μg/mL) | (μg/mL) | % E | (ug/mL) | (nm) | (nm) |
| 1 | 60 | N/A | 60 | 71.48 | 54.5 | 116.33 | 0.099 |
| 2 | N/A | 186 | 186 | 446.30 | 0.0 | 117.93 | 0.147 |
| 3 | 30 | 60 | 90 | 74.87 | 26.3 | 115.20 | 0.129 |
| 4 | 20 | 60 | 80 | 74.73 | 24.7 | 115.30 | 0.122 |
| 5 | 10 | 60 | 70 | 73.56 | 8.6 | 112.70 | 0.133 |
| 6 | 5 | 60 | 65 | 72.77 | 4.1 | 113.43 | 0.120 |
| 7 | 2 | 60 | 62 | 76.23 | 1.7 | 112.07 | 0.136 |
| 8 | 1 | 60 | 61 | 75.72 | 0.9 | 112.47 | 0.140 |
| 9 | 60 | 30 | 90 | 72.79 | 52.3 | 114.47 | 0.113 |
| 10 | 30 | 30 | 60 | 73.42 | 26.2 | 114.17 | 0.118 |
| 11 | 20 | 30 | 50 | 71.64 | 16.7 | 111.60 | 0.109 |
| 12 | 10 | 30 | 40 | 71.47 | 8.2 | 110.17 | 0.123 |
| 13 | 5 | 30 | 35 | 70.12 | 4.0 | 109.10 | 0.132 |
| 14 | 2 | 30 | 32 | 74.72 | 1.8 | 110.33 | 0.140 |
| 15 | 1 | 30 | 31 | 74.30 | 0.9 | 110.47 | 0.136 |
| 16 | 20 | 15 | 35 | 70.22 | 16.1 | 110.57 | 0.111 |
| 17 | 10 | 15 | 25 | 69.73 | 8.1 | 110.10 | 0.117 |
| 18 | 5 | 15 | 20 | 68.69 | 4.07 | 109.40 | 0.129 |
| 19 | 2 | 15 | 17 | 74.42 | 1.64 | 107.73 | 0.122 |
| 20 | 1 | 15 | 16 | 71.23 | 0.81 | 109.33 | 0.112 |
| Liq | 252 | N/A | 252 | 85.00 | 196 | 123.03 | 0.085 |
| control | | | | | | | |

It will be understood that the invention has been described by way of example only and modifications may be made whilst remaining within the scope and spirit of the invention.

1. A method of preparing a low-dose lyophilized RNA immunogenic composition, comprising:
forming a mixed aqueous composition comprising RNA containing lipid nanoparticles (RNA-LNPs), wherein said RNA encodes at least one immunogen, and empty lipid nanoparticles (empty LNPs); and
lyophilizing said mixed aqueous composition.

2. The method of claim 1, wherein said RNA is mRNA and said RNA-LNPs are mRNA-containing lipid nanoparticles (mRNA-LNPs).

3. The method of claim 1, wherein said RNA is self-amplifying mRNA (SAM) and said RNA-LNPs are SAM-containing lipid nanoparticles (SAM-LNPs).

4. The method of claim 3, wherein a composition containing said SAM-LNPs and a separate composition containing said empty LNPs are mixed to form said mixed aqueous composition and said mixed aqueous composition is thereafter lyophilized.

5. The method of claim 4, wherein said empty LNPs are present in an effective amount up to 25% dry weight of lipids based on the total dry weight of lipids in said empty LNPs and the total dry weight of lipids in said SAM-LNPs.

6. A vaccine composition comprising the immunogenic composition of claim 1.

7. A low-dose lyophilized immunogenic composition, comprising:
RNA containing lipid nanoparticles (RNA-LNPs), wherein said RNA encodes at least one immunogen; and
empty lipid nanoparticles (empty LNPs).

8. The low-dose lyophilized immunogenic composition of claim 7, wherein said RNA is mRNA and said RNA-LNPs are mRNA-containing lipid nanoparticles (mRNA-LNPs).

9. The low-dose lyophilized immunogenic composition of claim 7, wherein said RNA is self-amplifying mRNA (SAM) and said RNA-LNPs are SAM-containing lipid nanoparticles (SAM-LNPs).

10. The low-dose lyophilized immunogenic composition of claim 7, wherein said low-dose immunogenic composition can be reconstituted with a liquid for reconstitution to prepare an injectable vaccine composition that is ready for injection, without further dilution after reconstitution.

11. The low-dose lyophilized immunogenic composition of claim 9, having an amount of SAM of less than 60 μg of SAM that is encapsulated in LNPs to form SAM-LNPs, said amount of SAM being based on a single dose of a SAM-LNP low-dose lyophilized vaccine composition.

12. The low-dose lyophilized immunogenic composition of claim 10, having an amount of SAM of 1 to 6 μg of SAM that is encapsulated in LNPs to form SAM-LNPs, said amount of SAM being based on a single dose of a SAM-LNP low-dose lyophilized vaccine composition.

13. The low-dose lyophilized immunogenic composition of claim 7, wherein said low-dose lyophilized immunogenic composition comprises, in addition to components that form said LNPs and in addition to said RNA, one or more of the following additional dry components; buffer, sodium chloride and sugar.

14. The lyophilized immunogenic composition of claim 7, wherein said low-dose lyophilized vaccine comprises, in addition to components that form said LNPs and in addition to said RNA, a dry buffer component such that when the lyophilized vaccine composition is mixed with sterile water for injection an injectable aqueous pharmaceutically acceptable aqueous composition is formed.

15. A vaccine composition comprising the lyophilized immunogenic composition of claim 7.

16. An injectable vaccine composition made by reconstituting the vaccine composition of claim 15.

17. A method for making an injectable vaccine composition, comprising:
mixing a lyophilized vaccine composition comprising said immunogenic composition of claim 7 with an aqueous liquid for reconstitution to form a reconstituted composition, and optionally further diluting said reconstituted composition, to form an aqueous injectable vaccine composition.

18. A method for administering a low-dose vaccine to a subject, comprising:
reconstituting said lyophilized vaccine composition of claim 7 with a liquid for reconstitution to make a reconstituted composition; and
administering a low-dose vaccine comprising said reconstituted composition to said subject.

19. A method for administering a low-dose vaccine to a subject, comprising:
reconstituting said lyophilized vaccine composition of claim 15, with a liquid for reconstitution to make a reconstituted composition; and
administering a low-dose vaccine comprising said reconstituted composition to said subject without any further dilution after reconstitution.

20. A low-dose vaccine kit, comprising:
the lyophilized low-dose vaccine composition of claim 15 in a container; and
a sterile needle for injecting a vaccine composition and/or a second container containing a sterile aqueous solution and/or an adjuvant.

21. The low-dose vaccine kit of claim 20, wherein said container comprises enough of said lyophilized low-dose vaccine composition to make multiple low-doses of said low-dose vaccine.

22. A low-dose lyophilized RNA vaccine made by the method of claim 1.

23. The vaccine composition of claim 15, for use eliciting an immune response in a patient to which said vaccine composition is administered.

24. The injectable vaccine composition of claim 16, for use eliciting an immune response in a patient to which said vaccine composition is administered.

25. (canceled)
